SEQUENCE LISTING (14 sequences)

SEQ ID NO: 1 - protein, 9 aa, Spinacia oleracea
  REGION 1..9: note = motif
MAEIGYSVC

SEQ ID NO: 2 - protein, 6 aa, Spinacia oleracea
  REGION 1..6: note = motif
KWMCLR

SEQ ID NO: 3 - protein, 8 aa, Spinacia oleracea
  REGION 1..8: note = motif
HVGCVVDR

SEQ ID NO: 4 - protein, 9 aa, Spinacia oleracea
DQEDEGEDN

SEQ ID NO: 5 - DNA, 20 bp, Spinacia oleracea
  misc_feature 1..19: note = Reverse primer LRR domain Alpha
  source 1..20: mol_type = genomic DNA
acaagtggatgtgtcttagg

SEQ ID NO: 6 - DNA, 19 bp, Spinacia oleracea
  misc_feature 1..18: note = Forward primer LRR domain Beta
  source 1..19: mol_type = genomic DNA
ttcgccctcatcttcctgg

SEQ ID NO: 7 - DNA, 18 bp, Spinacia oleracea
  source 1..18: mol_type = genomic DNA
tcacgtgggttgtgttgt

SEQ ID NO: 8 - DNA, 1597 bp, Spinacia oleracea
  misc_feature 1..1597: note = Amplicon of LRR domain of the beta-WOLF 0 allele (Viroflay)
  source 1..1597: mol_type = genomic DNA
tcacgtgggttgtgttgtcgatagagatccagaaatagtctttttatgtagcaataagattcgttcgtatattagcggtcgctgcataaagaatccggtggattcacaaatagacaactggatgtgccttagggtgttggacttgtcagattcatgtgttaaagatttgtctgattcaataggtaagctgctgcacttaaggtatcttaacctctcttctaatataaagttggagataatccctgatgcaattacaagactgcataacttgcagacactacttttagaagattgcagaagtttaaaggagttgccaaaagatttttgcaaattggtcaaactgaggcacttggaattacagggttgtcatgatttgattggtatgtcatttggaatggataagctaactagtcttagaatactaccaaacattgtggtgggtaggaaggaacaaagtgttgatgatgagctgaaagccctaaaaggcctcaccgagataaaaggctccattgatatcacaatctattcaaaatatagaagagttgaaggcatgaatggcacaggaggaggagctgggtatttgaagagcatgaaacatctcacgggggttaatattacatttgatgaaggtggatgtgttaaccctgaagctgtgtatttgaagagcatgaaacatctcacgagggttattattatatttgattataaaggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgtccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcaccataatcggaaatcacggaataaataaagtgaagagactgtctggaagaacagggttggagcatttcactctgttggaatcactcaaactttcagatatagaagaccaggaagatgagggcgaa

SEQ ID NO: 9 - protein, 532 aa, Spinacia oleracea
  REGION 1..532: note = Amino acid sequence encoded by amplicon of LRR domain Beta Wolf 0(Viroflay)
HVGCVVDRDPEIVFLCSNKIRSYISGRCIKNPVDSQIDNWMCLRVLDLSDSCVKDLSDSIGKLLHLRYLNLSSNIKLEIIPDAITRLHNLQTLLLEDCRSLKELPKDFCKLVKLRHLELQGCHDLIGMSFGMDKLTSLRILPNIVVGRKEQSVDDELKALKGLTEIKGSIDITIYSKYRRVEGMNGTGGGAGYLKSMKHLTGVNITFDEGGCVNPEAVYLKSMKHLTRVIIIFDYKGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLTIIGNHGINKVKRLSGRTGLEHFTLLESLKLSDIEDQEDEGE

SEQ ID NO: 10 - DNA, 3456 bp, Spinacia oleracea
  misc_feature 1..1398: note = Coding sequence of the alpha-WOLF 23 allele
  source 1..3456: mol_type = genomic DNA
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgatgtatggaacgaggatcgtgagaagtggtttaaattggaagagttgttaatgttgggtcaagggggaagcaaggttgtagtgaccgcacgttcagagaagacagcaaatgtcatagggaaaagacatttttatacactggaatgtttgtcgccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataacaagattttgtcgatattgaagctcagttactacaatcttgcaaactctttgaagagttgttttagttattgtgcagtgtttcccaaggatcatgaaataaagaaggagatgttgattgaactttggatggcacaaggatatgttgtgccgttggatggaggtcaaagtatagaagatgctgccgaggaacattttgtaattttgttacgaaggtgtttctttcaagatgtagtgaaggatgaatacggtgatgttgattctgttaaaatccacgacttgatgcacgatgtcgcccaagaagtgggcagagaggaaatctgtatagtgaatgctaatacaaagaacttgggtgataaaatccgtcatgtacattgtgatgtcaatagatatgcacaaagagtctctctgtgtagccataagattcgttcgtatattggtggtcaatgtgaaaaacgttgggtggatacactaatagacaagtggatgtgtcttagggtgttggacttgtcaaggtcggatgttaaaaatttgcctaattcaataggtaaattgttgcacttgaggtgtcttaacctgtcttataatgatctgttgatactccctgatgcaattacaagactgcataatttgcagacactgcttttaaaagattgcggaagtttaatggagttgccaaaagatttttgcaaattggtcaaactgaggcacttggaattacagggttgtcatgatttgattggtatgccattgggaatggataggctaactagtcttagagtactgccattctttgtggtgggtaggaaggaacaaagtgttgatgatgagctgaaagccctaaaaggcctcaccgagataaaaggctccattcgtattagaatccattcaaagtatagaatagttgaaggcatgaatgacacaggaggagctgggtatttgaagagcatgaaacatctcacgggggttaatattagatttgatgatagagaaggtgtatttgttaaccctgaagctgtgttggcaaccctagagccaccttcaaatatcaagaggttagaggtgtggcattacgatggtacaacaattccagtatggggaagagcagagattaattgggcaatctccctctcgcatcttgttgacatcgagctttggcgttgtagtaatttgcaggagatgccagtgctgagtaaactgcctcatttgaaatcactgaaacttgaagatttgaataacttagagtacatggagagtagaagcagcagcagtagcagtgacacagaagcagcaacaccagaattaccaacattcttcccttcccttgaaaaacttacactttggcgtctgaacaagttgaagagttttgggaacaggagatcgagtagttttccccgcctctctgaattggaaatctgggaatgcccagatctaacgtggtttcctccctgtccaagccttgaagagttgacattgaaagacaaccatgaagcgttgcaaataatagtaaaaataacaacaacaagaggtaaagaagaaaaagaagaagacaagaatgctggtgttggaaattcacaagatgatgacaatgtcaaattacggaaggtggaaatagacaatctgggttatctcaaatcactgcccacaaattgtcttactcacctcgaccttacaataagtgattccaaggagggggagggtgaatgggaagttggggatgcatttcagaagtgtgtatcttctttgagaaagctcagcataatcggaaatcacggaataaataaagtgaagagactgtctggaagaacagggttggagcatttcactctgttggactcactcgaactttcaaatatagaagaccaggaagatgagggcgaagacaacatcatattctggaaatcctttcctcaaaacctccgcagtttggaaattgaagactctgacaaaatgacaagtttgcccatggggatgcagtacttaacctccctccaaaccctcgaactatcatattgtgatgaattgaattcccttccagaatggataagcagcttatcatctcttcaatacctgggcatattcaactgtccagccctgaaatcactaccagaagcaatgcggaacctcacctcccttcagacacttgggatatcggattgtccagacctagttaaaatatgcagaaaacccaacggcgaggactatcccaaaattcaatacatccccaaaattattatatggtag

SEQ ID NO: 11 - protein, 1151 aa, Spinacia oleracea
  REGION 1..466: note = Amino acid sequence of the alpha-WOLF 23 allele
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNDNEACSFLTIVGAGGLGKTALAQLVFNDERVKIEFHDLRYWVCVSDQDGGQFDVKEILCKILEVVTKEKVDNSSALELVQSQFQEKLRGKKYFLVLDDVWNEDREKWFKLEELLMLGQGGSKVVVTARSEKTANVIGKRHFYTLECLSPDYSWSLFEMSAFQKGHEQENHDELVDIGKKIVEKCYNNPLAITVVGSLLYGEEISKWRSFEMSELAKIGNGDNKILSILKLSYYNLANSLKSCFSYCAVFPKDHEIKKEMLIELWMAQGYVVPLDGGQSIEDAAEEHFVILLRRCFFQDVVKDEYGDVDSVKIHDLMHDVAQEVGREEICIVNANTKNLGDKIRHVHCDVNRYAQRVSLCSHKIRSYIGGQCEKRWVDTLIDKWMCLRVLDLSRSDVKNLPNSIGKLLHLRCLNLSYNDLLILPDAITRLHNLQTLLLKDCGSLMELPKDFCKLVKLRHLELQGCHDLIGMPLGMDRLTSLRVLPFFVVGRKEQSVDDELKALKGLTEIKGSIRIRIHSKYRIVEGMNDTGGAGYLKSMKHLTGVNIRFDDREGVFVNPEAVLATLEPPSNIKRLEVWHYDGTTIPVWGRAEINWAISLSHLVDIELWRCSNLQEMPVLSKLPHLKSLKLEDLNNLEYMESRSSSSSSDTEAATPELPTFFPSLEKLTLWRLNKLKSFGNRRSSSFPRLSELEIWECPDLTWFPPCPSLEELTLKDNHEALQIIVKITTTRGKEEKEEDKNAGVGNSQDDDNVKLRKVEIDNLGYLKSLPTNCLTHLDLTISDSKEGEGEWEVGDAFQKCVSSLRKLSIIGNHGINKVKRLSGRTGLEHFTLLDSLELSNIEDQEDEGEDNIIFWKSFPQNLRSLEIEDSDKMTSLPMGMQYLTSLQTLELSYCDELNSLPEWISSLSSLQYLGIFNCPALKSLPEAMRNLTSLQTLGISDCPDLVKICRKPNGEDYPKIQYIPKIIIW

SEQ ID NO: 12 - DNA, 1398 bp, Spinacia oleracea
  source 1..1398: mol_type = genomic DNA
tggatgtgtcttagggtgttggacttgtcaaggtcggatgttaaaaatttgcctaattcaataggtaaattgttgcacttgaggtgtcttaacctgtcttataatgatctgttgatactccctgatgcaattacaagactgcataatttgcagacactgcttttaaaagattgcggaagtttaatggagttgccaaaagatttttgcaaattggtcaaactgaggcacttggaattacagggttgtcatgatttgattggtatgccattgggaatggataggctaactagtcttagagtactgccattctttgtggtgggtaggaaggaacaaagtgttgatgatgagctgaaagccctaaaaggcctcaccgagataaaaggctccattcgtattagaatccattcaaagtatagaatagttgaaggcatgaatgacacaggaggagctgggtatttgaagagcatgaaacatctcacgggggttaatattagatttgatgatagagaaggtgtatttgttaaccctgaagctgtgttggcaaccctagagccaccttcaaatatcaagaggttagaggtgtggcattacgatggtacaacaattccagtatggggaagagcagagattaattgggcaatctccctctcgcatcttgttgacatcgagctttggcgttgtagtaatttgcaggagatgccagtgctgagtaaactgcctcatttgaaatcactgaaacttgaagatttgaataacttagagtacatggagagtagaagcagcagcagtagcagtgacacagaagcagcaacaccagaattaccaacattcttcccttcccttgaaaaacttacactttggcgtctgaacaagttgaagagttttgggaacaggagatcgagtagttttccccgcctctctgaattggaaatctgggaatgcccagatctaacgtggtttcctccctgtccaagccttgaagagttgacattgaaagacaaccatgaagcgttgcaaataatagtaaaaataacaacaacaagaggtaaagaagaaaaagaagaagacaagaatgctggtgttggaaattcacaagatgatgacaatgtcaaattacggaaggtggaaatagacaatctgggttatctcaaatcactgcccacaaattgtcttactcacctcgaccttacaataagtgattccaaggagggggagggtgaatgggaagttggggatgcatttcagaagtgtgtatcttctttgagaaagctcagcataatcggaaatcacggaataaataaagtgaagagactgtctggaagaacagggttggagcatttcactctgttggactcactcgaactttcaaatatagaagaccaggaagatgagggcgaa

SEQ ID NO: 13 - protein, 466 aa, Spinacia oleracea
WMCLRVLDLSRSDVKNLPNSIGKLLHLRCLNLSYNDLLILPDAITRLHNLQTLLLKDCGSLMELPKDFCKLVKLRHLELQGCHDLIGMPLGMDRLTSLRVLPFFVVGRKEQSVDDELKALKGLTEIKGSIRIRIHSKYRIVEGMNDTGGAGYLKSMKHLTGVNIRFDDREGVFVNPEAVLATLEPPSNIKRLEVWHYDGTTIPVWGRAEINWAISLSHLVDIELWRCSNLQEMPVLSKLPHLKSLKLEDLNNLEYMESRSSSSSSDTEAATPELPTFFPSLEKLTLWRLNKLKSFGNRRSSSFPRLSELEIWECPDLTWFPPCPSLEELTLKDNHEALQIIVKITTTRGKEEKEEDKNAGVGNSQDDDNVKLRKVEIDNLGYLKSLPTNCLTHLDLTISDSKEGEGEWEVGDAFQKCVSSLRKLSIIGNHGINKVKRLSGRTGLEHFTLLDSLELSNIEDQEDEGE

SEQ ID NO: 14 - DNA, 3462 bp, Spinacia oleracea
  source 1..3462: mol_type = genomic DNA
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtaggggaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgacgtatggaacgaggatcgtgagaagtggcttcgtttggaagagttgttaatgttgggtcaagggggaagcaaggttgtagtgaccacacgttctgagaaaacagcaaatgtcatagggaaaagacatttatatacactggaatgtttgtcaccagattattcatggagcttatttgaaatgtcggcttttcagaaagggcaggagcaggaaaaccatcacgaactagttcgtattgggaaaaagattgttgaaaaatgttataacaatccacttgtgataaccgtggttggaagtcttctttatggagaggagataagtaagtggcaatcatttgaaaggagtgagttggccaaaattggcaatggggataacaagattttgtcgatattgaagctcagttactacaatcttgcaaactctttgaagagttgttttagttattgtgcagtgtttcccaaggatcatgaaataaagaaggagatgttgattgaactttggatggcacaaggatatgttgtgccgttggatggaggtcaaagtatagaagatgctgccgaagaacattttgtaattttgttacgaaggtgtttctttcaagatgtaaagaaggatgaatatggtgatgtttatgctgttaaaatccacgacttgatgcacgatgtcgcccaagaagtggggagggaggaaatatgtgtagtgaatgataatacaaagaacttgggtgataaaatccgtcatgtacactgtgatgtcaatagatatgcacaaagagtctctctgtgtagccataagattcgttcgtatattggtggtaaatgtgaaaaacgttgggtggatacactaatagacaagtggatgtgtcttagggtgttggacttgtcaaggtcggatgttaaaaatttgcctaattcaataggtaaattgttgcacttgaggtgtcttaacctgtcttataataaagatctgttgatactccctgatgcaattacaagactgcataatttgcagacactgcttttaaaagattgcagaagtttaaaggagttgccaaaagatttttgcaaattggtcaaactgagacacttggatttaaggggttgtgatgatttgattggtatgccattgggaatggatatgctaactagtcttagagtactgccattctttgtggtgggtaggaaggaacaaagtgatgatgatgagctgaaagccctaaaaggcctcaccgagataaaaggcgacattgatatcaaaatctgttccaattatagaatagttgaaggcatgaatgacacaggaggagctgggtatttgaagagcatgaaacatctcacgggggttattattacatttgatgataatgaagatggatgtgttaaacctgaagctgtattggaaacccttgagccaccttcaaatatcaagaggttacttataggaaggtaccgtggtacaacaattccagtatggggaagagcagagattaattgggcaatctccctctcacatcttgtcgatatcacgcttatttattgttataatttgcaggagattccagtgctgagtaaactgcctcatttgaaattactggaactttataagttgattagcttagagtacatggagaatacaagcagaagcagtagcagtgacacagaagcagcaacactagaattaccaacattcttcccttcccttaaaaaacttacacttatcggtctgacaaagttgaagggtctggggaacaggagatcgagtagttttccccgcctctctaaattggtaatctgggaatgcccagatctaacgtggtttcctccttgtccaagccttgaaacgttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgacattacaataagagattccgaaggggaggagggtgaatgggaagttggggatacatttcagaagtgtgtatcttctttgagaagcctcgtcataatcggaaatcagggaataaataaagtgaatagactgtctggaagaacagggttggagcatttcactctgttggactcactcaaattttcagatatagacgaccaggaagatgagggcgaagacaacatcatgttctggaaatcctttcctcaaaacctccgcaatttggaaattaaatactctgacaaaatgacaagtttgcccatggggatgcagtacttaacctccctccaaaccatccatctttattgttgtggtaaattgaattccattccagaatggataagcagcttatcatctcttcaatccctgtacataggagaatgtccagccctaaaatcactaccagaagcaatgcagaacctcacctcccttcagagccttaggattttgggttgtccagacctaattgaaagatgcaaaaaacccaacggagaggactatcccaaaattcaacacatccccaaaattgaactatggcggtaa

(19) United States
(12) Patent Application Publication
(10) Pub. No.: US 2026/0240098 A1
(43) Pub. Date: Aug. 20, 2026

(54) CUCUMBER MOSAIC VIRUS AND PERONOSPORA RESISTANCE IN SPINACIA OLERACEA

(71) Applicant: RIJK ZWAAN ZAADTEELT EN ZAADHANDEL B.V., De Lier (NL)

(72) Inventors: Vincent Laurens Adrianus KOCK, De Lier (NL); Raoul Jacobus Johannes Maria FRIJTERS, De Lier (NL)

(21) Appl. No.: 19/663,923

(22) Filed: Apr. 30, 2026

Related U.S. Application Data
(63) Continuation-in-part of application No. 18/923,953, filed on Oct. 23, 2024.
Application No. 18/923,953 is a continuation-in-part of application No. PCT/EP2023/059918, filed on Apr. 17, 2023.

(30) Foreign Application Priority Data
Apr. 28, 2022 (WO) PCT/EP2022/061377

(51) Int. Cl. A01H 1/04; A01H 1/00; C07K 14/415; C12Q 1/6895

(52) U.S. Cl. CPC A01H 1/045; A01H 1/126; C07K 14/415; C12Q 1/6895; C12Q 2600/13

(57) ABSTRACT

A spinach plant having an allele designated alpha-WOLF 23 encoding a protein conferring resistance to downy mildew and Cucumber Mosaic Virus, wherein said allele has: a nucleotide sequence having a coding sequence having at least 97% sequence identity to SEQ ID NO: 10; a nucleotide sequence encoding a protein having an amino acid sequence having at least 93.5% sequence identity to SEQ ID NO: 11; a nucleotide sequence encoding an LRR domain, said nucleotide sequence having at least 94.5% sequence identity to SEQ ID NO: 12; or a nucleotide sequence encoding an LRR domain having an amino acid sequence having at least 93% sequence similarity to SEQ ID NO: 13; and the plant does not include a second alpha-WOLF allele having a nucleotide having a coding sequence according to SEQ ID NO: 14.

### RELATED APPLICATIONS AND INCORPORATION BY REFERENCE

This application is a continuation-in-part application of U.S. patent application Ser. No. 18/923,953, filed Oct. 23, 2024, which is a continuation-in-part application of international patent application Serial No. PCT/EP2023/059918 filed Apr. 17, 2023, which published as PCT Publication No. WO 2023/208632 on Nov. 2, 2024, which claims benefit of international patent application Serial No. PCT/EP2022/061377 filed Apr. 28, 2022.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created Apr. 16, 2026, is named 20260416 Sequence listing.xml and is 28,731 bytes in size.

### FIELD OF THE INVENTION

The invention relates to a spinach plant resistant against Cucumber Mosaic Virus (CMV) and downy mildew. The invention also relates to propagation material of said spinach plant, to a cell of said spinach plant, and to seed of said spinach plant. The invention further relates to a method of producing a spinach plant resistant to CMV and downy mildew and to the use of said plant in breeding to confer resistance to CMV and downy mildew.

### BACKGROUND OF THE INVENTION

Spinach (Spinacia oleracea L.) is a flowering plant from the Amaranthaceae family that is grown as a vegetable. The consumable parts of spinach are the leaves and petioles from the vegetative stage. Spinach is sold loose, bunched, in pre-packed bags, canned, or frozen. There are three basic types of spinach: industry-, fresh and Asiatic spinach. Within these types three different leaf types can be recognised: savoy, semi-savoy and smooth types. Savoy has crinkly and curly leaves. Flat or smooth leaf spinach has broad, smooth leaves. Semi-savoy is a variety with slightly crinkled leaves. The main market for spinach is baby-leaf. Baby spinach leaves are often of the flat-leaf variety and usually the harvested leaves are not longer than about eight centimeter. These tender, sweet leaves are sold loose rather than in bunches. They are often used in salads, but can also be lightly cooked.

Downy mildew (Peronospora effusa) is a major threat for spinach growers because it directly affects the harvested leaves. In spinach, downy mildew is caused by the oomycete Peronospora effusa (formerly known as Peronospora farinosa f. sp. spinaciae). Infection makes the leaves unsuitable for sale and consumption, as it manifests itself phenotypically as yellow lesions on the older leaves, and on the abaxial leaf surface a greyish fungal growth can be observed. The infection can spread very rapidly, and it can occur both in glasshouse cultivation and in soil cultivation. The optimal temperature for formation and germination of P. effusa spores is 9 to 12° C., and it is facilitated by a high relative humidity. When spores are deposited on a humid leaf surface they can readily germinate and infect the leaf. Fungal growth is optimal between 8 and 20° C. and a relative humidity of ≥80%, and within 6 and 13 days after infection mycelium growth can be observed. Oospores of P. effusa can survive in the soil for up to 3 years, or as mycelium in seeds or living plants.

To date 20 pathogenic races of spinach downy mildew (Pe, formerly known as Pfs) have been officially identified and characterized, and many new candidates are observed in the field. The 20 officially recognized races of Peronospora effusa, are designated Pe:1 to Pe:20 (Pe:1 to Pe:17 were formerly known as Pfs:1 to Pfs17; Irish et al. Phytopathol. Vol. 98 μg. 894-900, 2008; Plantum NL (Dutch association for breeding, tissue culture, production and trade of seed and young plants) press release, “Benoeming van Pfs: 14, een nieuwe fysio van valse meeldauw in spinazie”, Sep. 19, 2012; Report Jim Correl (Univ. Arkansas) and Steven Koike (UC Cooperative Extension, Monterey County), “Race Pfs: 14—Another new race of the spinach downy mildew pathogen”, Sep. 18, 2012; Plantum NL press release, “Denomination of Pfs: 15, a new race of downy mildew in spinach”, Sep. 2, 2014; Plantum NL press release, “Denomination of Pfs: 16, a new race of downy mildew in spinach, Mar. 15, 2016; Plantum NL press release, Denomination of Pfs: 17, a new race of downy mildew in spinach”, Apr. 16, 2018; Plantum NL press release, “Denomination of Pe: 18 and 19, two new races of downy mildew in spinach”, Apr. 15, 2021, Denomination of Pe: 20, a new race of downy mildew in spinach”, May 13, 2024).

The officially recognized Pe races are publicly available from Naktuinbouw, Sotaweg 22, 2371 GD Roelofarendsveen, the Netherlands, or via prof. Jim Correll (University of Arkansas, cppsi.ucdavis.edu/sites/g/files/dgvnsk8206/files/inline-files/Spinach_downy_mildew_Differentials_ver%20June%202021.pdf).

Especially the latest identified Peronospora races can break the resistance of many spinach varieties that are currently used commercially worldwide, and they thus pose a serious threat to the productivity of the spinach industry. Therefore, it is crucial to stay at the forefront of developments in this field, as Peronospora continuously develops the ability to break the resistances that are present in commercial spinach varieties. For this reason, new resistance genes against downy mildew are very valuable assets, and they form an important research focus in breeding and particular in spinach and lettuce breeding. One of the main goals of spinach breeders is to rapidly develop spinach varieties with a resistance to as many Peronospora races as possible, including the latest identified races, before these races become wide-spread and pose a threat to the industry.

In commercial spinach varieties, resistance against downy mildew is usually caused by so-called R-genes. R-gene mediated resistance is based on the ability of a plant to recognize the invading pathogen. In many cases this recognition occurs after the pathogen has established the first phases of interaction and transferred a so called pathogenicity (or avirulence) factor into the plant cell. These pathogenicity factors interact with host components in order to establish conditions which are favorable for the pathogen to invade the host and thereby cause disease. When a plant is able to recognize the events triggered by the pathogenicity factors a resistance response can be initiated. In many different plant pathogen interaction systems such as the interaction of spinach with different downy mildew strains, the plant initiates these events only after specific recognition of the invading pathogen.

Co-evolution of plant and pathogen has led to an arms race in which a R-gene mediated resistance is sometimes overcome as a consequence of the capability of the pathogen to interact with and modify alternative host targets or the same targets in a different way, such that the recognition is lost and infection can be established successfully resulting in disease. In order to re-establish resistance in a plant, a new R-gene has to be introduced which is able to recognize the mode of action of an alternative pathogenicity factor.

Despite the fact that the durability of R-genes is relatively low, in spinach R-genes are still the predominant form of defense against downy mildew. This is mainly due to the fact that it is the only form of defense that gives absolute resistance. So far plant breeders have been very successful in generating downy mildew resistant spinach varieties by making use of resistance genes residing in the wild germplasm of the crop species. Even though R-genes are extensively used in spinach breeding, until several years ago not much was known about these R-genes.

Rijk Zwaan found that the R-genes officially recognized in spinach are in fact all different alleles of two tightly linked genes, the alpha- and the beta-WOLF genes (WO2018/060474; Kock et al.). This was also the first time that R-genes, or better R-alleles, were characterized at the molecular level, i.e. their nucleotide and amino acid sequence was determined. Although this provides the breeder with tools that increase the efficiency of detecting and selecting R-alleles, adequately responding to newly emerging downy mildew races is still crucial for developing commercially successful spinach varieties.

Another disease threatening the production of spinach is Cucumber Mosaic Virus (CMV). Cucumber Mosaic Virus is a positive-sense single stranded RNA virus belonging to the Bromoviridae family and the Cucumovirus genus. The virus has a worldwide distribution, and is believed to have the broadest host range of any known plant virus. It has been reported to be able to infect over 1200 plant species in over 100 plant families.

The virus can be transmitted in many different ways e.g. mechanically, by insect vectors such as aphids, on seeds and even by parasitic weeds. The symptoms and severity of CMV infection depend on the species and the age of the plant. Typical symptoms of CMV infection are mottling, yellowing, the formation of ringspots, stunting and distortion of leaf, fruit and flowers.

CMV is in spinach also known as spinach blight. Symptoms observed on infected spinach plants commonly are leaf chlorotic mottle, narrowing, wrinkling and inward rolling of the leaves, and distortion of the veins.

A CMV resistance allele is already known in the art. However, this is an allele of the alpha-WOLF gene, whose alleles normally provide resistance to downy mildew. Since this particular allele of the alpha-WOLF gene called alpha-CMV does not provide any resistance to downy mildew, providing resistance to the wide array of downy mildew isolates becomes more complicated.

Therefore, it is the object of the invention to provide a new resistance allele conferring resistance to CMV in spinach and to provide molecular tools for identifying this resistance allele.

### SUMMARY OF THE INVENTION

The WOLF genes, which are either “alpha-WOLF” type or “beta-WOLF” type genes (together referred to as “the WOLF genes or alpha/beta-WOLF genes”) each encode a protein that belongs to the CCNBS-LRR (Coiled Coil-Nucleotide Binding Site-Leucine-Rich Repeat) family. Depending on the allelic variant (or the allelic variants) that is (are) present in a spinach plant, said plant will produce a variant of the WOLF protein that confers a certain resistance profile to pathogenic races of downy mildew (WO2018/060474; Kock et al.). There is also one variant known that provides resistance to CMV (WO2019/063839; Kock et al.) but does not provide resistance to downy mildew. The research leading to the present invention has now elucidated that the allelic variant designated alpha-WOLF 23 provides resistance to both downy mildew and CMV.

In the context of this invention the term “allele” or “allelic variant” is used to designate a version of a gene that is linked to a specific phenotype, i.e. resistance, more in particular CMV and downy mildew resistance. It was found that a spinach plant may carry one or two WOLF genes. For each of these two WOLF genes multiple alleles exist. The WOLF gene allele of the invention confers resistance to CMV and downy mildew.

The beta-WOLF gene of spinach variety Viroflay is located on scaffold12735 (sequence: GenBank: KQ143339.1), at position 213573-221884. In case the spinach plant also carries or only carries an alpha-WOLF gene, the alpha-WOLF gene is located at approximately the same location as where the beta-WOLF gene is located on scaffold12735 in the Viroflay genome assembly.

According to one aspect of the present invention, provided is a spinach plant comprising an allele of an alpha-WOLF gene encoding a protein which confers resistance to Cucumber Mosaic Virus (CMV), wherein the protein comprises in its amino acid sequence the motif MAEIGYSVC (SEQ ID NO: 1) at its N-terminus, and the motif KWMCLR (SEQ ID NO: 2); and wherein said allele comprises:
a) a nucleotide sequence comprising a coding sequence having at least 97% sequence identity to SEQ ID NO: 10, or
b) a nucleotide sequence encoding a protein having an amino acid sequence having at least 93.5% sequence identity to SEQ ID NO: 11, or
c) a nucleotide sequence encoding an LRR domain, wherein the nucleotide sequence has at least 94.5% sequence identity to SEQ ID NO: 12, or
d) a nucleotide sequence encoding an LRR domain, wherein the LRR domain comprises an amino acid sequence having at least 93% sequence similarity to SEQ ID NO: 13,
wherein said plant does not comprise a second alpha-WOLF allele having a nucleotide comprising a coding sequence according to SEQ ID NO: 14.

In some embodiments, the said allele further provides resistance to at least Peronospora effusa races Pe:3, Pfs:5, Pe:8, Pe:9, Pe:11, Pe:14, Pe:16 and Pe:19 when homozygously present in a spinach plant.

In some embodiments, the allele of the alpha-WOLF gene is as comprised in the genome of a spinach plant, representative sample of seed of which was deposited with the NCIMB under accession number NCIMB 44747.

In some embodiments, the allele of the alpha-WOLF gene comprises the nucleotide sequence comprising the coding sequence having at least 97% sequence identity to SEQ ID NO: 10.

In some embodiments, the allele of the alpha-WOLF gene comprises the nucleotide sequence encoding the protein which has the amino acid sequence having at least 93.5% sequence identity to SEQ ID NO: 11.

In some embodiments, the allele of the alpha-WOLF gene comprises the nucleotide sequence encoding the LRR domain, wherein the nucleotide sequence has at least 94.5% sequence identity to SEQ ID NO: 12.

In some embodiments, the allele of the alpha-WOLF gene comprises the nucleotide sequence encoding the LRR domain, wherein the LRR domain comprises the amino acid sequence having at least 93% sequence similarity to SEQ ID NO: 13.

In some embodiments, the spinach plant is an agronomically elite spinach plant. In some embodiments, the agronomically elite spinach plant is a hybrid variety or an inbred line.

In some embodiments, further provided is a seed of, or from, or that produces the spinach plant as described herein and comprises the allele of the alpha-WOLF gene encoding the protein which confers resistance to CMV. In some embodiments, further provided is a method for growing a spinach plant at least resistant to CMV, comprising the step of sowing or planting the seed as described herein, a representative sample of which was deposited with the NCIMB under accession number NCIMB 44747.

In some embodiments, further provided is propagation material from or suitable for producing spinach plant as described herein, wherein the propagation material is suitable for sexual reproduction, and a microspore, pollen, ovary, ovule, embryo sac or an egg cell, or the propagation material is suitable for vegetative reproduction and comprises a cutting, root, stem cell, or a protoplast, or the propagation material is suitable for tissue culture of regenerable cells or protoplasts and comprises a leaf, pollen, embryo, cotyledon, hypocotyl, meristematic cell, root, root tip, anther, flower or a stem, and wherein the propagation material comprises allele of the alpha-WOLF gene encoding the protein which confers resistance to CMV.

In another aspect of the present invention, provided is a method for producing a spinach plant resistant to CMV comprising the steps of:
a) crossing a first parent plant, wherein the first parent plant is the spinach plant as described herein, with a second 1 parent plant to obtain an F1 population;
b) optionally performing one or more rounds of selfing and/or crossing with a plant from the F1 population to obtain a further generation population;
c) selecting from the first or the further generation population a plant resistant to CMV.

In another aspect of the present invention, a method for producing a hybrid spinach seed resistant to CMV, comprising the steps of crossing a first parent plant with a second parent plant, wherein one or both parent plants are homozygous for the allele of the alpha-WOLF gene as described herein and harvesting the hybrid seed is also provided.

In some embodiments, further provided is hybrid is as produced by the methods described herein. Also provided is a plant grown from the hybrid seed as described herein.

In another aspect of the present invention, a method for selecting a spinach plant resistant to Cucumber Mosaic Virus (CMV) is provided, the method comprising
a) identifying by genetic analysis the presence of an allele of the alpha-WOLF gene encoding a protein which confers resistance to CMV; and
b) selecting a plant that comprises said allele of the alpha-WOLF gene;
wherein:
   the allele of the alpha-WOLF gene comprises:
i) a nucleotide sequence comprising a coding sequence having at least 97% sequence identity to SEQ ID NO: 10; or
ii) a nucleotide sequence encoding a protein which has an amino acid sequence having at least 93.5% sequence identity to SEQ ID NO: 11; or
iii) a nucleotide sequence encoding an LRR domain wherein the nucleotide sequence has at least 94.5% sequence identity to SEQ ID NO: 12; or
iv) a nucleotide sequence encoding an LRR domain, wherein the LRR domain comprises an amino acid sequence having at least 93% sequence similarity to SEQ ID NO: 13;
wherein the protein comprises in its amino acid sequence the motif MAEIGYSVC (SEQ ID NO: 1) at its N-terminus, and the motif KWMCLR (SEQ ID NO: 2); and
wherein the plant selected in b) does not comprise a second alpha-WOLF allele having a nucleotide comprising a coding sequence according to SEQ ID NO: 14.

In some embodiments, the method of producing the hybrid spinach seed resistant to CMV further comprises the steps of:
c) further testing the selected plant for CMV resistance; and
d) selecting the further tested plant that exhibits CMV resistance.

In yet another aspect of the present invention, a method for identifying a spinach plant comprising an allele of an alpha-WOLF gene encoding a protein which confers resistance to Cucumber Mosaic Virus (CMV) is also provided, wherein:
the allele of the alpha-WOLF gene comprises:
i) a nucleotide sequence comprising a coding sequence having at least 97% sequence identity to SEQ ID NO: 10 (nucleotide sequence (i)); or
ii) a nucleotide sequence encoding a protein which has an amino acid sequence having at least 93.5% sequence identity to SEQ ID NO: 11 (nucleotide sequence (ii)); or
iii) a nucleotide sequence encoding an LRR domain wherein the nucleotide sequence has at least 94.5% sequence identity to SEQ ID NO: 12 (nucleotide sequence (iii)); or
iv) a nucleotide sequence encoding an LRR domain, wherein the LRR domain comprises an amino acid sequence having at least 93% sequence identity to SEQ ID NO: 13 (nucleotide sequence (iv)); and
the protein encoded by the allele of the alpha-WOLF gene comprises in its amino acid sequence the motif MAEIGYSVC (SEQ ID NO: 1) at its N-terminus, and the motif KWMCLR (SEQ ID NO: 2);
the method comprises:
detecting by genetic analysis in the genome of the spinach plant nucleotide (i), or nucleotide (ii), nucleotide (iii), or nucleotide (iv), or a unique polymorphism in the allele of the alpha-WOLF gene, thereby identifying the spinach plant comprising the allele of the alpha-WOLF gene;
and optionally testing the plant identified as comprising the allele of the alpha-WOLF gene for exhibiting resistance to CMV;
wherein the plant identified by the method does not comprise a second alpha-WOLF allele having a nucleotide comprising a coding sequence according to SEQ ID NO: 14.

In some embodiments of the method for identifying the spinach plant comprising the allele of an alpha-WOLF gene encoding the protein which confers resistance to CMV, the method comprises determining the presence of the nucleotide sequence encoding the LRR domain by using a primer pair comprising a forward primer and a reverse primer to amplify the nucleotide sequence encoding the LRR domain, wherein the forward primer is a nucleic acid molecule having the sequence of SEQ ID NO: 5 and the reverse primer is a nucleic acid molecule having the sequence of SEQ ID NO: 6.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description

### Deposit

The Deposits with NCIMB Ltd, Wellheads Place, Dyce, Aberdeen AB21 7 GB, UK, on Mar. 6, 2026 under deposit accession number NCIMB 44747 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary, including if the deposit ever becomes unviable, during that period.

### DETAILED DESCRIPTION

A genome assembly for spinach variety Viroflay—which is susceptible to all known pathogenic races of Peronospora effusa—is publicly available (Spinacia oleracea cultivar Syn Viroflay, whole genome shotgun sequencing project; Bioproject: PRJNA41497; GenBank: AYZV00000000.2; BioSample: SAMN02182572, see also Dohm et al, 2014, Nature 505:546-549). In this genome assembly for Viroflay, the beta-WOLF gene is located on scaffold12735 (sequence: GenBank: KQ143339.1), at position 213573-221884. The sequence covered by this interval comprises the entire genomic sequence of the beta-WOLF gene of Viroflay, plus 2000 basepairs sequence upstream from the gene, plus the sequence downstream from the gene, up to the locus of the neighbouring gene that is situated downstream from the WOLF gene. Spinach variety Viroflay only possesses a single WOLF gene, namely a beta-WOLF gene, but most other spinach lines harbor a single alpha-type WOLF gene at the same location in the genome. Other spinach lines harbor two WOLF genes at approximately the same location in the genome. In such cases, the two WOLF genes are positioned adjacent to each other. In most spinach lines that harbor two WOLF genes, one of said WOLF genes belongs to the alpha-type, and the other WOLF gene belongs to the beta-type. It was observed that this allelic variation in the WOLF locus is responsible for differences in resistance to pathogenic races of Peronospora effusa.

The difference between an allele of an alpha-WOLF gene and an allele of a beta-WOLF gene lies in the presence of specific conserved amino acid motifs in the encoded protein sequence. As mentioned above, all WOLF proteins possess—from N- to C-terminus—the following domains that are generally known in the art: a coiled coil domain (RX-CC-like, cd14798), an NBS domain (also referred to as “NB-ARC domain”, pfam00931; van der Biezen & Jones, 1998, Curr. Biol. 8: R226-R228), and leucine-rich repeats (IPR032675) which encompass the LRR domain. In addition, all WOLF proteins comprise in their amino acid sequence the motif “MAEIGYSVC” (SEQ ID NO: 1) at the N-terminus. In addition to this, all alpha-WOLF proteins comprise the motif “KWMCLR” (SEQ ID NO: 2) in their amino acid sequence, whereas all beta-WOLF proteins comprise the motif “HVGCVVDR” (SEQ ID NO: 3) in their amino acid sequence.

The present invention provides a new CMV and downy mildew resistance conferring allele of the alpha-WOLF gene, herein referred to as the ‘allele of the invention’ or alpha-WOLF 23 allele, encoding a protein which confers resistance to Peronospora effusa and CMV when expressed in a spinach plant.

The alpha-WOLF 23 allele encodes a CC-NBS-LRR protein which confers resistance to CMV and Peronospora effusa races Pe:3, Pfs:5, Pe:8, Pe:9, Pe:11, Pe:14, Pe:16 and Pe:19 and does not confer resistance to Peronospora effusa races Pe:2, Pe:4, Pe:6, Pe:7, Pe:10, Pe:13, Pe:15, Pe:18, Pe:20 when expressed homozygously in a spinach plant, and wherein the protein comprises in its amino acid sequence the motif “MAEIGYSVC” (SEQ ID NO: 1) at its N-terminus, and the motif “KWMCLR” (SEQ ID NO: 2); and wherein said allele comprises:
a) a nucleotide sequence comprising a coding sequence that has at least 97.0%, 97.5%, 98.0%, 98.5%, 99.0%, 99.5%, 100% sequence identity to SEQ ID NO: 10, or
b) a nucleotide sequence encoding a protein which has an amino acid sequence that has at least 93.5%, 94.0%, 94.5%, 95.0%, 95.5%, 96.0%, 96.5%, 97.0%, 97.5%, 98.0%, 98.5%, 99.0%, 99.5%, 100% sequence similarity to SEQ ID NO: 11, or
c) a nucleotide sequence encoding an LRR domain which nucleotide sequence has at least 94.5% 95.0%, 95.5%, 96.0%, 96.5%, 97.0%, 97.5%, 98.0%, 98.5%, 99.0%, 99.5%, 100% sequence identity to SEQ ID NO: 12, or
d) a nucleotide sequence encoding an LRR domain which has an amino acid sequence that has at least 93%, 93.5%, 94.0%, 94.5%, 95.0%, 95.5%, 96.0%, 96.5%, 97.0%, 97.5%, 98.0%, 98.5%, 99.0%, 99.5%, 100% sequence similarity to SEQ ID NO: 13,

Optionally, the alpha-WOLF 23 allele further comprises an additional motif in its amino acid sequence, namely “DQEDEGEDN” (SEQ ID NO: 4).

The allele of the invention is a nucleic acid, in particular a nucleic acid molecule, more in particular an isolated nucleic acid molecule.

The allele of the invention encodes a CC-NBS-LRR protein, wherein the protein comprises in its amino acid sequence the motif “MAEIGYSVC” at its N-terminus, and the motif “KWMCLR”, and wherein the allele comprises a coding sequence that in order in order of increased preference has at least 97.0%, 97.5%, 98.0%, 98.5%, 99.0%, 99.5%, 100% sequence identity to SEQ ID NO: 10.

Preferably, the allele of the invention encodes a CC-NBS-LRR protein, wherein the protein comprises in its amino acid sequence the motif “MAEIGYSVC” at its N-terminus, and the motif “KWMCLR”, and wherein the allele comprises a coding sequence that in order in order of increased preference has at least 97.0% sequence identity to SEQ ID NO: 10.

In a preferred embodiment, the allele of the invention encodes a CC-NBS-LRR protein, wherein the protein comprises in its amino acid sequence the motif “MAEIGYSVC” at its N-terminus, and the motif “KWMCLR”, and wherein the allele comprises a coding sequence according to SEQ ID NO: 10.

The allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding a protein which has an amino acid sequence that in order of increased preference has at least 93.5%, 94.0%, 94.5%, 95.0%, 95.5%, 96.0%, 96.5%, 97.0%, 97.5%, 98.0%, 98.5%, 99.0%, 99.5%, 100% sequence similarity to SEQ ID NO: 11.

Preferably, the allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding a protein which has an amino acid sequence that in order of increased preference has at least 93.5% sequence similarity to SEQ ID NO: 11.

In a preferred embodiment, the allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding a protein having an amino acid sequence according to SEQ ID NO: 11.

The allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding an LRR domain which nucleotide sequence in order of increased preference has at least 94.5% 95.0%, 95.5%, 96.0%, 96.5%, 97.0%, 97.5%, 98.0%, 98.5%, 99.0%, 99.5%, 100% sequence identity to SEQ ID NO: 12.

Preferably, the allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding an LRR domain which nucleotide sequence in order of increased preference has at least 94.5% sequence identity to SEQ ID NO: 12.

In a preferred embodiment, the allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding an LRR domain which has a nucleotide sequence according to SEQ ID NO: 12.

The allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding an LRR domain which has an amino acid sequence that in order of increased preference has at least 93%, 93.5%, 94.0%, 94.5%, 95.0%, 95.5%, 96.0%, 96.5%, 97.0%, 97.5%, 98.0%, 98.5%, 99.0%, 99.5%, 100% sequence similarity to SEQ ID NO: 13.

Preferably, the allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding an LRR domain which has an amino acid sequence that in order of increased preference has at least 93% sequence similarity to SEQ ID NO: 13.

In a preferred embodiment, the allele of the invention encodes a CC-NBS-LRR protein, wherein the protein comprises in its amino acid sequence the motif “MAEIGYSVC” at its N-terminus, and the motif “KWMCLR”, and wherein the allele comprises a nucleotide sequence encoding an LRR domain which has an amino acid sequence according to SEQ ID NO: 13.

The alpha-WOLF 23 allele when homozygously present in a spinach plant confers resistance to CMV, and complete resistance to at least Peronospora effusa races Pe:3, Pfs:5, Pe:8, Pe:9, Pe:11, Pe:14, Pe:16, and Pe:19 and does not confer resistance to at least Peronospora effusa races Pe:2, Pe:4, Pe:6, Pe:7, Pe:10, Pe:13, Pe:15, Pe:18 and Pe:20.

The alpha-WOLF 23 allele when heterozygously present in a spinach plant confers resistance to CMV, and complete resistance to at least Peronospora effusa races Pe:3, Pfs:5, Pe:9, Pe:11, Pe:14 and Pe:19, intermediate resistance to Pe:8 and Pe:16 and does not confer resistance to at least Peronospora effusa races Pe:2, Pe:4, Pe:6, Pe:7, Pe:10, Pe:13, Pe:15, Pe:18 and Pe:20.

The invention further relates to a protein encoded by the allele of the invention. This protein is also referred to herein as the “protein of the invention” and confers CMV and downy mildew resistance to a spinach plant, in particular to at least Peronospora effusa races Pe:3, Pfs:5, Pe:8, Pe:9, Pe:11, Pe:14, Pe:16, and Pe:19 when the alpha-WOLF 23 encoding the protein of the invention is homozygously present in the genome of the spinach plant.

As used herein, sequence identity is the percentage of nucleotides or amino acids that is identical between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity score should be obtained. The percentage sequence identity is calculated through comparison over the length of the shortest sequence in the assessment. In the present case, a nucleotide sequence represents a gene that at least comprises a start codon and a stop codon or encodes an amino acid sequence which comprises a complete protein encoded by such a gene.

Sequence similarity for an amino acid sequence is calculated using EMBOSS stretcher 6.6.0 (www.ebi.ac.uk/jdispatcher/psa/emboss_stretcher), using the EBLOSUM62 matrix with settings Gap open penalty: 12 and Gap extend penalty: 2. In case of DNA, sequence similarity is calculated using the DNA full matrix with settings Gap open penalty: 16 and Gap extend penalty: 4.

The invention further relates to a plant, preferably a plant of the species Spinacia oleracea L., wherein the plant comprises the allele of the invention either heterozygously or homozygously in its genome. A plant comprising the allele of the invention in its genome is referred to herein as a ‘plant of the invention’. In the context of this invention, a plant of the species Spinacia oleracea L. is a spinach plant.

In a further embodiment, the plant of the invention is an agronomically elite spinach plant.

In the context of this invention, an agronomically elite plant is a plant having a genotype that, as a result of human intervention, comprises an accumulation of distinguishable and desirable agronomic traits which allow a producer to harvest a product of commercial significance, preferably the agronomically elite plant of the invention is a plant of an inbred line or a hybrid. Such an agronomically elite plant can also be referred to as a cultivated spinach plant.

As used herein, a plant of an inbred line is a plant of a population of plants that is the result of three or more rounds of selfing, or backcrossing; or which plant is a double haploid. An inbred line may e.g. be a parent line used for the production of a commercial hybrid.

As used herein, a hybrid plant is a plant which is the result of a cross between two different plants having different genotypes. More in particular, a hybrid plant is the result of a cross between plants of two different inbred lines. Such a hybrid plant may e.g. be a plant of an F1 hybrid variety.

Seed of Spinacia oleracea L. comprising the alpha-WOLF 23 allele was deposited with the NCIMB under accession number NCIMB 44747.

The invention thus relates to plants grown from seed deposited under NCIMB accession number NCIMB 44747.

The alpha-WOLF 23 allele when present in a spinach plant confers resistance to CMV, preferably complete resistance to CMV. Resistance against CMV in spinach plants may be tested by mechanically inoculating plants with the CMV virus which is a mixture of two isolates (NL 16 and SP 43) when the plant has two or three true leaves. Plants to be tested are grown under a regime with a day temperature of 20° C. and a night temperature of 18° C. and receive at least 16 hours of light. Inoculation is done by dusting all true leaves of the plants with carborundrum powder and subsequently rubbing them with a sponge soaked with inoculum. The inoculum is a mixture of equal amounts of both isolates diluted in water, preferably a 1:10 dilution. After inoculation, plants may be slightly rinsed with water. Symptoms may be observed 7 to 9 days after inoculation. A resistant plant, i.e. a plant comprising the allele of the invention, shows no symptoms, while a susceptible plant typically shows dwarf growth and mosaic symptoms in the heart of the plant. A detailed example of the test described herein can be found in the CPVO protocol for tests on distinctness, uniformity, and stability for spinach as available on: cpvo.europa.eu/sites/default/files/documents/2025-10/spinacia-5.5-corr.pdf.

The resistance of a spinach plant against one or more races of Peronospora effusa can be determined using a seedling test. Herein, a seedling test is defined as a test wherein spinach plants are planted in trays containing growth medium, fertilized twice a week after seedling emergence. Plants are inoculated at the first true leaf stage with a sporangial suspension having a concentration of approximately 2.5×10⁵/ml of one of the pathogenic races of Peronospora effusa or isolates to be tested. Thirty plants per race are tested. The inoculated plants are placed in a dew chamber at 18° C. with 100% relative humidity for a 24 h period, and then moved to a growth chamber at 18° C. with a 12 h photoperiod for 6 days. After 6 days, the plants are returned to the dew chamber for 24 h to induce sporulation, and subsequently scored for a disease reaction.

As used herein, a plant is completely resistant against a Peronospora effusa race when a plant shows no symptoms in the seedling test described herein.

As used herein, a plant is intermediately resistant against a Peronospora effusa race when a plant shows only symptoms of chlorosis, or sporulation occurring only on the tips of the cotyledons in the seedling test described herein.

As used herein, a plant is susceptible to an isolate of a Peronospora effusa race when a plant shows more than only symptoms of chlorosis, or when sporulation occurs on an area larger than only the tips of the cotyledons in the seedling test described herein.

A plant carrying the alpha-WOLF 23 allele in heterozygous form may further comprise a beta-WOLF 0 allele on the complementary chromosome (as e.g. present in variety Viroflay) wherein the beta-WOLF 0 allele does not confer any resistance to downy mildew or CMV. Alternatively, a plant heterozygous for the alpha-WOLF 23 allele may further comprise an allele of the alpha or beta-WOLF gene on the complementary chromosome that does provide resistance to downy mildew. Preferably, such an allele would complement the alpha-WOLF 23 allele such that the spinach plant will be at least intermediately resistant to one or more other races to which the alpha-WOLF 23 allele does not provide resistance. Most preferably the other allele of the alpha or beta-WOLF gene complements the alpha-WOLF 23 allele such that the plant is resistant to Peronospora effusa races Pe:1 to Pe:20. In one embodiment such a plant is an agronomically elite plant.

Alternatively, the resistance profile of a plant carrying the alpha-WOLF 23 allele is complemented by a resistance conferring allele of a totally different gene. Examples of such genes are e.g. DMR1 as described in U.S. Pat. No. 8,354,570, DMR6 as described in U.S. Pat. No. 9,121,029, p10 as described in U.S. Pat. No. 10,226,016, and a locus from Spinacia tetrandra on chromosome 4 as described in U.S. Pat. No. 12,365,913.

In one embodiment the plant of the invention does not comprise an alpha-WOLF 10 allele having SEQ ID NO: 14 on the complementary chromosome.

The invention thus relates to a spinach plant carrying the alpha-WOLF 23 allele, and further comprising another genetic determinant, together resulting in resistance against Peronospora effusa races Pe:1 to Pe:20 and resistance to CMV. The genetic determinant can be another resistance conferring alpha/beta-WOLF allele and/or a resistance conferring allele of a totally different gene.

Another aspect of the invention relates to a seed capable of growing into a plant of the invention wherein said plant comprises the allele of the invention. The invention also relates to use of said seed for the production of a plant of the invention, by growing said seed into a plant.

Yet another aspect of the invention relates to a leaf harvested from a spinach plant of the invention either in natural or processed form.

The invention also relates to propagation material suitable for producing a plant of the invention, wherein the propagation material is suitable for sexual reproduction, and is in particular selected from a microspore, a pollen, an ovary, an ovule, an embryo sac and an egg cell, or is suitable for vegetative reproduction, and is in particular selected from a cutting, a root, a stem a cell, and a protoplast, or is suitable for tissue culture of regenerable cells or protoplasts, and is in particular selected from a leaf, a pollen, an embryo, a cotyledon, a hypocotyl, a meristematic cell, a root, a root tip, an anther, a flower, a seed and a stem, wherein the propagation material comprises the allele of the invention.

The invention further relates to a cell of a plant of the invention. Such a cell may either be in isolated form, or a part of the complete plant or parts thereof and still forms a cell of the invention because such a cell comprises the allele of the invention. A cell of the invention may also be a regenerable cell that can regenerate into a new plant of the invention.

The invention further relates to plant tissue of a plant of the invention, which comprises the allele of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention further relates to a method for the production of a plant comprising the allele of the invention, which plant is resistant to Peronospora effusa, by using tissue culture or by using vegetative propagation.

Progeny of a plant, a cell, a tissue, or a seed of the invention, which progeny comprises the alpha-WOLF 23 allele also part of the invention. Such progeny can in itself be a plant, a cell, a tissue, or a seed. The progeny can in particular be progeny of a plant of the invention, representative seeds of which were deposited under NCIMB number 44747. As used herein, progeny comprises the first and all further descendants from a cross with a plant of the invention, wherein a cross comprises a cross with itself or a cross with another plant, and wherein a descendant that is determined to be progeny comprises the allele of the invention. Descendants can be obtained through selfing and/or further crossing of the deposit. Progeny also encompasses material that is obtained by vegetative propagation or another form of multiplication.

The invention further relates to the germplasm of plants of the invention. The germplasm is constituted by all inherited characteristics of an organism and according to the invention encompasses at least the resistance trait of the invention. The germplasm can be used in a breeding program for the development of plants that show resistance to Peronospora effusa. The use of germplasm that comprises the allele of the invention in breeding is also part of the present invention. Seed capable of growing into a plant comprising the allele of the invention and being representative for the germplasm was deposited with the NCIMB under accession number NCIMB 44747.

The invention also relates to the use of the alpha-WOLF 23 allele for producing a spinach plant that is resistant to Peronospora effusa and CMV.

The current invention also relates to the use of a plant of the invention as a crop, as a source of seed or as a source of propagation material.

The invention also relates to the use of a plant of the invention in breeding to confer resistance to Peronospora effusa and CMV.

The invention further relates to a method for seed production comprising growing a spinach plant from a seed of the invention that comprises the allele of the invention homozygously, allowing the plant to produce seed and harvesting the seed. Production of the seed is suitably done by selfing or by crossing with another plant that is optionally also a plant of the invention. The plant grown from the seed produced as described herein is resistant to CMV and resistant to Peronospora effusa races Pe:3, Pfs:5, Pe:8, Pe:9, Pe:11, Pe:14, Pe:16, and Pe:19.

The invention also relates to a method for producing a hybrid spinach seed, comprising crossing a first parent plant with a second parent plant and harvesting the resultant hybrid spinach seed, wherein the first parent plant and/or the second parent plant is a plant of the invention. Preferably, at least one of the parent plants comprises the allele of the invention homozygously.

In a particular embodiment, the first and/or second parent plant is a plant of an inbred line as defined herein.

The invention also relates to the hybrid seed produced by the method described herein and a hybrid plant grown from said hybrid seed, wherein said hybrid seed and plant comprise the allele of the invention.

Transgenic techniques used for transferring nucleotide sequences between plants that are sexually incompatible can also be used to produce a plant of the invention, by transferring the allele of the invention from one species to another. Techniques that can suitably be used comprise general plant transformation techniques known to the skilled person, such as the use of an Agrobacterium-mediated transformation method. A plant of the deposit or a descendant thereof is a suitable source of the modified gene.

The invention further relates to a method for identifying a spinach plant comprising the allele of the invention, wherein the method comprises the following steps:
a1) detecting the allele of the invention in the genome of a plant by determining the sequence of the allele, or
a2) detecting the allele of the invention by determining the sequence of the LRR domain of the allele of the invention in the genome of a plant, or
a3) by detecting a unique polymorphism in the allele of the invention, and
b) optionally, the method may further comprise testing of the plant comprising the allele of the invention for exhibiting resistance to CMV and/or Peronospora effusa.

The LRR domain of the allele of the invention can be determined by using a primer pair to amplify the LRR domain, wherein the forward primer is a nucleic acid molecule having the sequence of SEQ ID NO: 5 and wherein the reverse primer is a nucleic acid molecule having the sequence of SEQ ID NO: 6.

The invention further relates to a method for selecting a spinach plant resistant to CMV, comprising identifying the presence of the allele of the invention, optionally testing the plant for resistance against CMV, and selecting a plant comprising said allele as a plant which is resistant to CMV.

Introduction of the allele of the invention can also be done through introgression from a plant comprising said allele, for example from a plant, representative seed of which was deposited as NCIMB 44747, or from progeny thereof, or from any other plant of the invention. Breeding methods such as crossing and selection, backcrossing, recombinant selection, or other breeding methods that result in the transfer of a genetic sequence from a resistant plant to a susceptible plant can be used. A resistant plant can be of the same species or of a different and/or wild species. Difficulties in crossing between species can be overcome through techniques known in the art such as embryo rescue, or cis-genesis can be applied instead. Progeny of a deposit can be sexual or vegetative descendants of that deposit, which can be selfed and/or crossed, and can be of an F1, F2, or further generation as long as the descendants of the deposit still comprise the modified allele the invention as present in seed of that deposit. A plant produced by such method is also a part of the invention.

In one embodiment the invention relates to a spinach plant or a progeny plant thereof in which the alpha-WOLF 23 allele including its regulatory elements is introduced via cisgenesis. The cisgenic introduction of the Alpha-WOLF 23 allele including its regulatory elements can be done via traditional transformation techniques e.g. Agrobacterium-mediated transformation, or by CRISPR/Cas-assisted techniques e.g. base editing, prime editing, and HDR.

The skilled artisan knows how to identify the regulatory elements upstream and downstream of the coding sequence as well as the introns of an alpha-WOLF gene and the alpha-WOLF 23 allele in particular.

In one embodiment the invention relates to a spinach plant or progeny thereof in which the Alpha-WOLF 23 allele is located on another chromosome than chromosome 3, preferably on a part of the genome where it does not lead to interruptions of any endogenous genes.

The invention also relates to a method for the production of a plant resistant to CMV and Peronospora effusa races Pe:3, Pfs:5, Pe:8, Pe:9, Pe:11, Pe:14, Pe:16 and Pe:19, comprising the steps of:
a) crossing a first parent plant comprising the allele of the invention with a second parent plant to obtain an F1 population;
b) optionally performing one or more rounds of selfing and/or crossing with a plant from the F1 population to obtain a further generation;
c) selecting from the F1 population or further generation population a plant that comprises the allele of the invention as a resistant plant.

The invention also relates to a method for producing a plant which is resistant to CMV and Peronospora effusa races Pe:3, Pfs:5, Pe:8, Pe:9, Pe:11, Pe:14, Pe:16 and Pe:19, said method comprising:
a) crossing a first parent plant comprising the allele of the invention with a second parent plant not comprising the allele of the invention;
b) backcrossing the plant resulting from step a) with the second parent plant for at least three generations;
c) selecting from the third or higher backcross population a plant that comprises at least the allele of the invention of the first parent plant of step a) as the plant which is resistant to CMV and Peronospora effusa.

The invention additionally provides for a method of introducing another desired trait into a plant that is resistant to CMV and Peronospora effusa, comprising:
a) crossing a plant comprising the allele of the invention with a second plant that comprises the other desired trait to produce F1 progeny;
b) selecting in the F1 for a plant that comprises the resistance and the other desired trait;
c) crossing the optionally selected F1 progeny with one of the parents for at least three generations, to produce backcross progeny;
d) selecting backcross progeny comprising the resistance and the other desired trait; and
e) optionally repeating steps c) and d) one or more times in succession to produce selected fourth or higher backcross progeny that comprises the resistances and the other desired trait.

Optionally, selfing steps are performed after any of the crossing or backcrossing steps in above-described methods. Selection of a plant comprising the CMV and Peronospora effusa resistances and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, improved shelf-life, water stress and heat stress tolerance, and male sterility. The invention includes a plant produced by this method.

The present invention will be further illustrated in the Examples that follow and that are for illustration purposes only. The Examples are not intended to limit the invention in any way. In the Examples and in the application, reference is made to the following figures.

### Resistance Information

The downy mildew resistance profile conferred by the alpha-WOLF 23 allele when heterozygously or homozygously present in a spinach plant. A “−” means complete resistance against a particular downy mildew race; “(−)” means intermediate resistance against a particular downy mildew race; “+” means that the allele confers no resistance and would cause a plant only carrying the alpha-WOLF 23 allele to be fully susceptible for that particular downy mildew race; “−*” means that when the allele is present homozygously it confers complete resistance against a particular downy mildew race, while the allele does confer intermediate resistance to that particular downy mildew race when present heterozygously; “nt” means that it has not been tested against that isolate.

TABLE 1
alpha-WOLF 23 resistance profile

| Peronospora effusa | Resistance |
|---|---|
| race | score |
| Pe: 1 | nt |
| Pe: 2 | + |
| Pe: 3 | − |
| Pe: 4 | + |
| Pe: 5 | − |
| Pe: 6 | + |
| Pe: 7 | + |
| Pe: 8 | −* |
| Pe: 9 | − |
| Pe: 10 | + |
| Pe: 11 | − |
| Pe: 12 | nt |
| Pe: 13 | + |
| Pe: 14 | − |
| Pe: 15 | + |
| Pe: 16 | −* |
| Pe: 17 | nt |
| Pe: 18 | + |
| Pe: 19 | − |
| Pe: 20 | + |

### Sequence Information

TABLE 2
Sequence information.

| | |
|---|---|
| SEQ ID NO: 1: | MAEIGYSVC |
| SEQ ID NO: 2: | KWMCLR |
| SEQ ID NO: 3: | HVGCVVDR |
| SEQ ID NO: 4: | DQEDEGEDN |
| SEQ ID NO: 5: | ACAAGTGGATGTGTCTTAGG |
| Forward primer | |
| LRR domain | |
| (Alpha) | |
| SEQ ID NO: 6: | TTCGCCCTCATCTTCCTGG |
| Reverse primer | |
| LRR domain | |
| (Alpha and | |
| Beta) | |
| SEQ ID NO: 7: | TCACGTGGGTTGTGTTGT |
| Forward primer | |
| LRR domain | |
| (Beta) | |
| SEQ ID NO: 8: | TCACGTGGGTTGTGTTGTCGATAGAGATCCAGAAATAGTCTT |
| Amplicon of | TTTATGTAGCAATAAGATTCGTTCGTATATTAGCGGTCGCTG |
| LRR domain of | CATAAAGAATCCGGTGGATTCACAAATAGACAACTGGATGT |
| the beta-WOLF | GCCTTAGGGTGTTGGACTTGTCAGATTCATGTGTTAAAGATT |
| 0 allele | TGTCTGATTCAATAGGTAAGCTGCTGCACTTAAGGTATCTTA |
| (Viroflay) | ACCTCTCTTCTAATATAAAGTTGGAGATAATCCCTGATGCAA |
| | TTACAAGACTGCATAACTTGCAGACACTACTTTTAGAAGATT |
| | GCAGAAGTTTAAAGGAGTTGCCAAAAGATTTTTGCAAATTG |
| | GTCAAACTGAGGCACTTGGAATTACAGGGTTGTCATGATTTG |
| | ATTGGTATGTCATTTGGAATGGATAAGCTAACTAGTCTTAGA |
| | ATACTACCAAACATTGTGGTGGGTAGGAAGGAACAAAGTGT |
| | TGATGATGAGCTGAAAGCCCTAAAAGGCCTCACCGAGATAA |
| | AAGGCTCCATTGATATCACAATCTATTCAAAATATAGAAGA |
| | GTTGAAGGCATGAATGGCACAGGAGGAGGAGCTGGGTATTT |
| | GAAGAGCATGAAACATCTCACGGGGGTTAATATTACATTTG |
| | ATGAAGGTGGATGTGTTAACCCTGAAGCTGTGTATTTGAAG |
| | AGCATGAAACATCTCACGAGGGTTATTATTATATTTGATTAT |
| | AAAGGTGGATGTGTTAACCCTGAAGCTGTGTTGGCAACCCT |
| | AGAGCCACCTTCAAATATCAAGAGGTTAGAGATGTGGCATT |
| | ACAGTGGTACAACAATTCCAGTATGGGGAAGAGCAGAGATT |
| | AATTGGGCAATCTCCCTCTCACATCTTGTCGACATCACGCTT |
| | GAAGATTGTTACAATTTGCAGGAGATGCCAGTGCTGAGTAA |
| | ACTGCCTCATTTGAAATCACTGGAACTTACAGAGTTGGATAA |
| | CTTAGAGTACATGGAGAGTAGAAGCAGCAGCAGTAGCAGTG |
| | ACACAGAAGCAGCAACACCAGAATTACCAACATTCTTCCCT |
| | TCCCTTGAAAAACTTACACTTTGGCGTCTGGACAAGTTGAAG |
| | GGTTTTGGGAACAGGAGATCGAGTAGTTTTCCCCGCCTCTCT |
| | AAATTGGAAATCTGGAAATGTCCAGATCTAACGTCATTTCCT |
| | TCTTGTCCAAGCCTTGAAGAGTTGGAATTGAAAGAAAACAA |
| | TGAAGCGTTGCAAATAATAGTAAAAATAACAACAACAAGAG |
| | GTAAAGAAGAAAAAGAAGAAGACAAGAATGCTGGTGTTGG |
| | AAATTCACAAGATGATGACAATGTCAAATTATGGAAGGTGG |
| | AAATAGACAATCTGGGTTATCTCAAATCACTGCCCACAAATT |
| | GTCTGACTCACCTCGACCTTACAATAAGTGATTCCAAGGAGG |
| | GGGAGGGTGAATGGGAAGTTGGGGATGCATTTCAGAAGTGT |
| | GTATCTTCTTTGAGAAGCCTCACCATAATCGGAAATCACGGA |
| | ATAAATAAAGTGAAGAGACTGTCTGGAAGAACAGGGTTGGA |
| | GCATTTCACTCTGTTGGAATCACTCAAACTTTCAGATATAGA |
| | AGACCAGGAAGATGAGGGCGAA |
| SEQ ID NO: 9: | HVGCVVDRDPEIVFLCSNKIRSYISGRCIKNPVDSQIDNWMCLR |
| Amino acid | VLDLSDSCVKDLSDSIGKLLHLRYLNLSSNIKLEIIPDAITRLHNL |
| sequence | QTLLLEDCRSLKELPKDFCKLVKLRHLELQGCHDLIGMSFGMD |
| encoded by | KLTSLRILPNIVVGRKEQSVDDELKALKGLTEIKGSIDITIYSKYR |
| amplicon of | RVEGMNGTGGGAGYLKSMKHLTGVNITFDEGGCVNPEAVYL |
| LRR domain | KSMKHLTRVIIIFDYKGGCVNPEAVLATLEPPSNIKRLEMWHYS |
| Beta Wolf 0 | GTTIPVWGRAEINWAISLSHLVDITLEDCYNLQEMPVLSKLPHL |
| (Viroflay) | KSLELTELDNLEYMESRSSSSSSDTEAATPELPTFFPSLEKLTLW |
| | RLDKLKGFGNRRSSSFPRLSKLEIWKCPDLTSFPSCPSLEELELK |
| | ENNEALQIIVKITTTRGKEEKEEDKNAGVGNSQDDDNVKLWK |
| | VEIDNLGYLKSLPTNCLTHLDLTISDSKEGEGEWEVGDAFQKC |
| | VSSLRSLTIIGNHGINKVKRLSGRTGLEHFTLLESLKLSDIEDQE |
| | DEGE |
| SEQ ID NO: | ATGGCCGAAATCGGATACTCGGTTTGTGCGAAACTCATCGA |
| 10: Coding | AGTGATTGGCAGTGAGCTGATCAAAGAGATTTGCGACACAT |
| sequence of the | GGGGTTACAAATCTCTTCTTGAGGACCTCAACAAAACTGTAT |
| alpha-WOLF 23 | TGACGGTCAGGAACGTTCTCATTCAGGCCGGGGTGATGCGG |
| allele | GAGCTTACTAGTGAACAACAAGGTTTCATTGCAGACCTTAA |
| | AGATGTTGTTTATGATGCTGATGACTTGTTCGACAAGTTACT |
| | CACTCGTGCTGAGCGAAAACAGATTGATGGAAACGAAATCT |
| | CTGAAAAGGTACGTCGTTTCTTTTCCTCTAGTAACAAGATCG |
| | GTCAAGCTTACTACATGTCTCGTAAGGTTAAGGAAATTAAG |
| | AAGCAGTTGGATGAAATTGTTGATAGGCATACAAAATTTGG |
| | GTTTAGTGCTGAGTTTATACCTGTTTGTAGGGAAAGGGGGAA |
| | CGAGAGGGAAACACGTTCATATATAGATGTCAAGAATATTC |
| | TTGGGAGGGATAAAGATAAGAATGATATCATAGATAGGTTG |
| | CTTAATCGTAATGATAATGAAGCTTGTAGTTTCCTGACCATA |
| | GTGGGAGCGGGAGGATTGGGAAAAACTGCTCTTGCCCAACT |
| | TGTGTTCAATGATGAAAGGGTCAAAATTGAGTTTCATGATTT |
| | GAGGTATTGGGTTTGTGTCTCTGATCAAGATGGGGGCCAATT |
| | TGATGTGAAAGAAATCCTTTGTAAGATTTTAGAGGTGGTTAC |
| | TAAGGAGAAAGTTGATAATAGTTCCGCATTGGAATTGGTAC |
| | AAAGCCAATTTCAAGAGAAGTTAAGAGGAAAGAAGTACTTC |
| | CTTGTTCTTGATGATGTATGGAACGAGGATCGTGAGAAGTG |
| | GTTTAAATTGGAAGAGTTGTTAATGTTGGGTCAAGGGGGAA |
| | GCAAGGTTGTAGTGACCGCACGTTCAGAGAAGACAGCAAAT |
| | GTCATAGGGAAAAGACATTTTTATACACTGGAATGTTTGTCG |
| | CCAGATTATTCATGGAGCTTATTTGAAATGTCGGCTTTTCAG |
| | AAAGGGCATGAGCAGGAAAACCATGACGAACTAGTTGATAT |
| | TGGGAAAAAGATTGTTGAAAAATGTTATAACAATCCACTTG |
| | CTATAACGGTGGTAGGAAGTCTTCTTTATGGAGAGGAGATA |
| | AGTAAGTGGCGGTCATTTGAAATGAGTGAGTTGGCCAAAAT |
| | TGGCAATGGGGATAACAAGATTTTGTCGATATTGAAGCTCA |
| | GTTACTACAATCTTGCAAACTCTTTGAAGAGTTGTTTTAGTT |
| | ATTGTGCAGTGTTTCCCAAGGATCATGAAATAAAGAAGGAG |
| | ATGTTGATTGAACTTTGGATGGCACAAGGATATGTTGTGCCG |
| | TTGGATGGAGGTCAAAGTATAGAAGATGCTGCCGAGGAACA |
| | TTTTGTAATTTTGTTACGAAGGTGTTTCTTTCAAGATGTAGTG |
| | AAGGATGAATACGGTGATGTTGATTCTGTTAAAATCCACGA |
| | CTTGATGCACGATGTCGCCCAAGAAGTGGGCAGAGAGGAAA |
| | TCTGTATAGTGAATGCTAATACAAAGAACTTGGGTGATAAA |
| | ATCCGTCATGTACATTGTGATGTCAATAGATATGCACAAAGA |
| | GTCTCTCTGTGTAGCCATAAGATTCGTTCGTATATTGGTGGT |
| | CAATGTGAAAAACGTTGGGTGGATACACTAATAGACAAGTG |
| | GATGTGTCTTAGGGTGTTGGACTTGTCAAGGTCGGATGTTAA |
| | AAATTTGCCTAATTCAATAGGTAAATTGTTGCACTTGAGGTG |
| | TCTTAACCTGTCTTATAATGATCTGTTGATACTCCCTGATGCA |
| | ATTACAAGACTGCATAATTTGCAGACACTGCTTTTAAAAGAT |
| | TGCGGAAGTTTAATGGAGTTGCCAAAAGATTTTTGCAAATTG |
| | GTCAAACTGAGGCACTTGGAATTACAGGGTTGTCATGATTTG |
| | ATTGGTATGCCATTGGGAATGGATAGGCTAACTAGTCTTAGA |
| | GTACTGCCATTCTTTGTGGTGGGTAGGAAGGAACAAAGTGTT |
| | GATGATGAGCTGAAAGCCCTAAAAGGCCTCACCGAGATAAA |
| | AGGCTCCATTCGTATTAGAATCCATTCAAAGTATAGAATAGT |
| | TGAAGGCATGAATGACACAGGAGGAGCTGGGTATTTGAAGA |
| | GCATGAAACATCTCACGGGGGTTAATATTAGATTTGATGATA |
| | GAGAAGGTGTATTTGTTAACCCTGAAGCTGTGTTGGCAACCC |
| | TAGAGCCACCTTCAAATATCAAGAGGTTAGAGGTGTGGCAT |
| | TACGATGGTACAACAATTCCAGTATGGGGAAGAGCAGAGAT |
| | TAATTGGGCAATCTCCCTCTCGCATCTTGTTGACATCGAGCT |
| | TTGGCGTTGTAGTAATTTGCAGGAGATGCCAGTGCTGAGTAA |
| | ACTGCCTCATTTGAAATCACTGAAACTTGAAGATTTGAATAA |
| | CTTAGAGTACATGGAGAGTAGAAGCAGCAGCAGTAGCAGTG |
| | ACACAGAAGCAGCAACACCAGAATTACCAACATTCTTCCCT |
| | TCCCTTGAAAAACTTACACTTTGGCGTCTGAACAAGTTGAAG |
| | AGTTTTGGGAACAGGAGATCGAGTAGTTTTCCCCGCCTCTCT |
| | GAATTGGAAATCTGGGAATGCCCAGATCTAACGTGGTTTCCT |
| | CCCTGTCCAAGCCTTGAAGAGTTGACATTGAAAGACAACCA |
| | TGAAGCGTTGCAAATAATAGTAAAAATAACAACAACAAGAG |
| | GTAAAGAAGAAAAAGAAGAAGACAAGAATGCTGGTGTTGG |
| | AAATTCACAAGATGATGACAATGTCAAATTACGGAAGGTGG |
| | AAATAGACAATCTGGGTTATCTCAAATCACTGCCCACAAATT |
| | GTCTTACTCACCTCGACCTTACAATAAGTGATTCCAAGGAGG |
| | GGGAGGGTGAATGGGAAGTTGGGGATGCATTTCAGAAGTGT |
| | GTATCTTCTTTGAGAAAGCTCAGCATAATCGGAAATCACGG |
| | AATAAATAAAGTGAAGAGACTGTCTGGAAGAACAGGGTTGG |
| | AGCATTTCACTCTGTTGGACTCACTCGAACTTTCAAATATAG |
| | AAGACCAGGAAGATGAGGGCGAAGACAACATCATATTCTGG |
| | AAATCCTTTCCTCAAAACCTCCGCAGTTTGGAAATTGAAGAC |
| | TCTGACAAAATGACAAGTTTGCCCATGGGGATGCAGTACTT |
| | AACCTCCCTCCAAACCCTCGAACTATCATATTGTGATGAATT |
| | GAATTCCCTTCCAGAATGGATAAGCAGCTTATCATCTCTTCA |
| | ATACCTGGGCATATTCAACTGTCCAGCCCTGAAATCACTACC |
| | AGAAGCAATGCGGAACCTCACCTCCCTTCAGACACTTGGGA |
| | TATCGGATTGTCCAGACCTAGTTAAAATATGCAGAAAACCC |
| | AACGGCGAGGACTATCCCAAAATTCAATACATCCCCAAAAT |
| | TATTATATGGTAG |
| SEQ ID NO: | MAEIGYSVCAKLIEVIGSELIKEICDTWGYKSLLEDLNKTVLTV |
| 11: Amino acid | RNVLIQAGVMRELTSEQQGFIADLKDVVYDADDLFDKLLTRAE |
| sequence of the | RKQIDGNEISEKVRRFFSSSNKIGQAYYMSRKVKEIKKQLDEIV |
| alpha-WOLF 23 | DRHTKFGFSAEFIPVCRERGNERETRSYIDVKNILGRDKDKNDII |
| allele | DRLLNRNDNEACSFLTIVGAGGLGKTALAQLVENDERVKIEFH |
| | DLRYWVCVSDQDGGQFDVKEILCKILEVVTKEKVDNSSALELV |
| | QSQFQEKLRGKKYFLVLDDVWNEDREKWFKLEELLMLGQGG |
| | SKVVVTARSEKTANVIGKRHFYTLECLSPDYSWSLFEMSAFQK |
| | GHEQENHDELVDIGKKIVEKCYNNPLAITVVGSLLYGEEISKWR |
| | SFEMSELAKIGNGDNKILSILKLSYYNLANSLKSCFSYCAVFPK |
| | DHEIKKEMLIELWMAQGYVVPLDGGQSIEDAAEEHFVILLRRC |
| | FFQDVVKDEYGDVDSVKIHDLMHDVAQEVGREEICIVNANTK |
| | NLGDKIRHVHCDVNRYAQRVSLCSHKIRSYIGGQCEKRWVDTL |
| | IDKWMCLRVLDLSRSDVKNLPNSIGKLLHLRCLNLSYNDLLILP |
| | DAITRLHNLQTLLLKDCGSLMELPKDFCKLVKLRHLELQGCHD |
| | LIGMPLGMDRLTSLRVLPFFVVGRKEQSVDDELKALKGLTEIK |
| | GSIRIRIHSKYRIVEGMNDTGGAGYLKSMKHLTGVNIRFDDREG |
| | VFVNPEAVLATLEPPSNIKRLEVWHYDGTTIPVWGRAEINWAIS |
| | LSHLVDIELWRCSNLQEMPVLSKLPHLKSLKLEDLNNLEYMES |
| | RSSSSSSDTEAATPELPTFFPSLEKLTLWRLNKLKSFGNRRSSSFP |
| | RLSELEIWECPDLTWFPPCPSLEELTLKDNHEALQIIVKITTTRG |
| | KEEKEEDKNAGVGNSQDDDNVKLRKVEIDNLGYLKSLPTNCL |
| | THLDLTISDSKEGEGEWEVGDAFQKCVSSLRKLSIIGNHGINKV |
| | KRLSGRTGLEHFTLLDSLELSNIEDQEDEGEDNIIFWKSFPQNLR |
| | SLEIEDSDKMTSLPMGMQYLTSLQTLELSYCDELNSLPEWISSL |
| | SSLQYLGIFNCPALKSLPEAMRNLTSLQTLGISDCPDLVKICRKP |
| | NGEDYPKIQYIPKIIIW |
| SEQ ID NO: | TGGATGTGTCTTAGGGTGTTGGACTTGTCAAGGTCGGATGTT |
| 12: Amplicon | AAAAATTTGCCTAATTCAATAGGTAAATTGTTGCACTTGAGG |
| of LRR domain | TGTCTTAACCTGTCTTATAATGATCTGTTGATACTCCCTGATG |
| of the alpha- | CAATTACAAGACTGCATAATTTGCAGACACTGCTTTTAAAAG |
| WOLF 23 allele | ATTGCGGAAGTTTAATGGAGTTGCCAAAAGATTTTTGCAAAT |
| | TGGTCAAACTGAGGCACTTGGAATTACAGGGTTGTCATGATT |
| | TGATTGGTATGCCATTGGGAATGGATAGGCTAACTAGTCTTA |
| | GAGTACTGCCATTCTTTGTGGTGGGTAGGAAGGAACAAAGT |
| | GTTGATGATGAGCTGAAAGCCCTAAAAGGCCTCACCGAGAT |
| | AAAAGGCTCCATTCGTATTAGAATCCATTCAAAGTATAGAAT |
| | AGTTGAAGGCATGAATGACACAGGAGGAGCTGGGTATTTGA |
| | AGAGCATGAAACATCTCACGGGGGTTAATATTAGATTTGAT |
| | GATAGAGAAGGTGTATTTGTTAACCCTGAAGCTGTGTTGGCA |
| | ACCCTAGAGCCACCTTCAAATATCAAGAGGTTAGAGGTGTG |
| | GCATTACGATGGTACAACAATTCCAGTATGGGGAAGAGCAG |
| | AGATTAATTGGGCAATCTCCCTCTCGCATCTTGTTGACATCG |
| | AGCTTTGGCGTTGTAGTAATTTGCAGGAGATGCCAGTGCTGA |
| | GTAAACTGCCTCATTTGAAATCACTGAAACTTGAAGATTTGA |
| | ATAACTTAGAGTACATGGAGAGTAGAAGCAGCAGCAGTAGC |
| | AGTGACACAGAAGCAGCAACACCAGAATTACCAACATTCTT |
| | CCCTTCCCTTGAAAAACTTACACTTTGGCGTCTGAACAAGTT |
| | GAAGAGTTTTGGGAACAGGAGATCGAGTAGTTTTCCCCGCC |
| | TCTCTGAATTGGAAATCTGGGAATGCCCAGATCTAACGTGGT |
| | TTCCTCCCTGTCCAAGCCTTGAAGAGTTGACATTGAAAGACA |
| | ACCATGAAGCGTTGCAAATAATAGTAAAAATAACAACAACA |
| | AGAGGTAAAGAAGAAAAAGAAGAAGACAAGAATGCTGGTG |
| | TTGGAAATTCACAAGATGATGACAATGTCAAATTACGGAAG |
| | GTGGAAATAGACAATCTGGGTTATCTCAAATCACTGCCCAC |
| | AAATTGTCTTACTCACCTCGACCTTACAATAAGTGATTCCAA |
| | GGAGGGGGAGGGTGAATGGGAAGTTGGGGATGCATTTCAGA |
| | AGTGTGTATCTTCTTTGAGAAAGCTCAGCATAATCGGAAATC |
| | ACGGAATAAATAAAGTGAAGAGACTGTCTGGAAGAACAGG |
| | GTTGGAGCATTTCACTCTGTTGGACTCACTCGAACTTTCAAA |
| | TATAGAAGACCAGGAAGATGAGGGCGAA |
| SEQ ID NO: | WMCLRVLDLSRSDVKNLPNSIGKLLHLRCLNLSYNDLLILPDAI |
| 13: Amino acid | TRLHNLQTLLLKDCGSLMELPKDFCKLVKLRHLELQGCHDLIG |
| sequence | MPLGMDRLTSLRVLPFFVVGRKEQSVDDELKALKGLTEIKGSIR |
| encoded by | IRIHSKYRIVEGMNDTGGAGYLKSMKHLTGVNIRFDDREGVFV |
| amplicon of the | NPEAVLATLEPPSNIKRLEVWHYDGTTIPVWGRAEINWAISLSH |
| alpha-WOLF 23 | LVDIELWRCSNLQEMPVLSKLPHLKSLKLEDLNNLEYMESRSSS |
| allele | SSSDTEAATPELPTFFPSLEKLTLWRLNKLKSFGNRRSSSFPRLS |
| | ELEIWECPDLTWFPPCPSLEELTLKDNHEALQIIVKITTTRGKEE |
| | KEEDKNAGVGNSQDDDNVKLRKVEIDNLGYLKSLPTNCLTHL |
| | DLTISDSKEGEGEWEVGDAFQKCVSSLRKLSIIGNHGINKVKRL |
| | SGRTGLEHFTLLDSLELSNIEDQEDEGE |

### EXAMPLES

### Example 1

### Testing for Resistance to CMV in Spinach Plants

The resistance to CMV infection was assayed as described in the CPVO protocol for tests on distinctness, uniformity, and stability for spinach as available on: cpvo.europa.eu/sites/default/files/documents/2025-10/spinacia-5.5-corr.pdf.

Spinach seeds of the invention together with seeds of varieties Viroflay and Polka were sown in 5×5 cm soil blocks and under a cultivation regime with a day temperature of 20° C. and a night temperature of 18° C. and receiving at least 16 hours of light. When the plants had developed three true leaves they were inoculated with CMV. Inoculation was done by dusting the leaves of the plants with carborundrum powder and subsequently rubbing the leaves with a sponge soaked in inoculum. The inoculum was a dilution (1:10) of isolates NL 16 and SP 43 in water. After inoculation plants were slightly rinsed with water.

Symptoms may be observed 7 to 9 days after inoculation. A resistant plant, i.e. a plant comprising the allele of the invention, shows no symptoms, while a susceptible plant typically shows dwarf growth and mosaic symptoms in the heart of the plant.

Plants for this specific test were scored as resistant or susceptible based on the development of symptoms. Plants exhibiting no symptoms were in this specific test considered as resistant. Plants that showed symptoms of infection were in this test scored a susceptible.

All plants of varieties Viroflay and Polka were scored as susceptible since they showed symptoms such as dwarf growth and mosaic symptoms in the heart of the plant, while plants of the invention showed no symptoms and were scored resistant.

### Example 2

Testing for Resistance to Peronospora effusa in Spinach Plants

The resistance to downy mildew infection was assayed as described by Irish et al. (2008; Phytopathol. 98:894-900), using the differential set of the International Seed Federation (worldseed.org/document/differential-sets-peronospora-effusa-pe-spinach/). Spinach plants of the invention were sown along with spinach plants from different other genotypes (see Table 3) in trays containing Scotts Redi-Earth medium, and fertilized twice a week after seedling emergence with Osmocote Peter's (13-13-13) fertilizer (Scotts). Plants were inoculated with a sporangial suspension (2.5×10⁵/ml) of a pathogenic race of Peronospora effusa at the first true leaf stage. In this manner, 4 officially recognized pathogenic race were tested.

The inoculated plants were placed in a dew chamber at 18° C. with 100% relative humidity for a 24 h period, and then moved to a growth chamber at 18° C. with a 12 h photoperiod for 6 days. After 6 days, the plants were returned to the dew chamber for 24 h to induce sporulation, and they were scored for disease reaction.

The resistance to downy mildew infection was assayed as described by Irish et al. (2008; Phytopathol. 98:894-900), using the differential set of the International Seed Federation (worldseed.org/document/differential-sets-peronospora-effusa-pe-spinach/). Spinach plants of the invention were grown along with spinach plants from different other genotypes (see Table 3) in trays containing Scotts Redi-Earth medium, and fertilized twice a week after seedling emergence with Osmocote Peter's (13-13-13) fertilizer (Scotts). Plants were inoculated with a sporangial suspension (2.5×10⁵/ml) of a pathogenic race of Peronospora effusa at the first true leaf stage. In this manner, 4 officially recognized pathogenic race were tested.

The inoculated plants were placed in a dew chamber at 18° C. with 100% relative humidity for a 24 h period, and then moved to a growth chamber at 18° C. with a 12 h photoperiod for 6 days. After 6 days, the plants were returned to the dew chamber for 24 h to induce sporulation, and they were scored for disease reaction.

Plants for this specific test were scored as resistant, intermediately resistant, or susceptible based on symptoms of chlorosis and signs of pathogen sporulation on the cotyledons and true leaves, as described by Irish et al. (2007; Plant Dis. 91:1392-1396). Plants exhibiting no evidence of chlorosis and sporulation were in this specific test considered as resistant. Resistant plants were re-inoculated to assess whether plants initially scored as resistant had escaped infection, or whether they were truly resistant. Plants that showed only symptoms of chlorosis, or sporulation occurring only on the tips of the cotyledons were scored as intermediately resistant. Plants showing more than these symptoms of downy mildew infection were scored as being susceptible.

Table 1 shows the resistance of a plant carrying the alpha-WOLF 23 allele to each one of these pathogenic races. Table 3 shows the differential set of spinach downy mildew races and the resistance of various spinach varieties (hybrids) to each one of these pathogenic races. A susceptible reaction is scored as “+” (indicating a successful infection by the fungus, with sporulation occurring on the entire cotyledon), and resistance is depicted as “−” (absence of sporulation on the cotyledons). A weak resistance response is indicated as “(−)”, which in practice means a slightly reduced level of infection (with only symptoms of chlorosis, or sporulation only occurring on the tips of the cotyledons in the differential seedling test).

TABLE 3

| Races/plants | Viroflay | NIL 5 | NIL 3 | NIL 4 | NIL 6 | NIL 1 | NIL 2 | NIL 9 | Caladonia | Meerkat | Hydrus | Yakalo |
|---|---|---|---|---|---|---|---|---|---|---|---|---|
| Pe: 1 | + | − | − | − | − | − | − | − | − | − | − | − |
| Pe: 2 | + | − | + | − | + | − | − | − | − | − | − | − |
| Pe: 3 | + | + | − | − | − | − | − | − | − | − | − | − |
| Pe: 4 | + | + | + | − | − | − | − | − | − | − | − | + |
| Pe: 5 | + | + | − | + | − | − | − | − | − | − | − | − |
| Pe: 6 | + | + | + | + | + | − | − | − | − | − | − | − |
| Pe: 7 | + | + | + | + | − | − | − | − | − | − | − | − |
| Pe: 8 | + | + | − | + | + | + | − | − | − | − | − | − |
| Pe: 9 | + | + | − | + | + | − | − | − | − | − | − | − |
| Pe: 10 | + | + | + | + | + | + | − | − | − | − | − | − |
| Pe: 11 | + | + | − | + | − | − | + | − | − | − | − | − |
| Pe: 12 | + | + | − | + | + | + | + | − | − | − | − | − |
| Pe: 13 | + | + | + | + | − | (−) | + | − | − | − | − | − |
| Pe: 14 | + | + | − | + | + | + | + | + | − | − | − | − |
| Pe: 15 | + | + | + | − | − | − | − | + | + | − | − | − |
| Pe: 16 | + | + | − | + | − | − | + | + | − | + | − | − |
| Pe: 17 | + | + | + | + | + | + | + | + | + | (−) | − | − |
| Pe: 18 | + | + | + | + | − | − | + | + | + | + | − | − |
| Pe: 19 | + | + | − | + | + | + | + | + | − | + | + | − |
| Pe: 20 | + | + | + | + | − | − | − | − | − | − | − | + |

### Example 3

### Amplification of the LRR Domain-Encoding Region

The isolated genomic DNA of a spinach plant comprising the alpha-WOLF 23 allele, of which a representative sample of seed was deposited with the NCIMB under accession number NCIMB 44747 was used in polymerase chain reactions (PCR), using forward primer ACAAGTGGATGTGTCTTAGG (SEQ ID NO: 5) and reverse primer TTCGCCCTCATCTTCCTGG (SEQ ID NO: 6). The primer pair amplifies the LRR domain-encoding region of an alpha-WOLF gene, and has been designed for selectively amplifying part of a WOLF gene, and not of other CC-NBS-LRR protein-encoding genes.

PCR conditions for amplifying the LRR domain-encoding region of an alpha-WOLF gene using primers having SEQ ID NO: 5 and SEQ ID NO: 6 were as follows, using Platinum Taq enzyme (Thermo Fisher Scientific):
3 minutes at 95° C. (initial denaturing step)
40 amplification cycles, each cycle consisting of: 30 seconds denaturation at 95° C., 30 seconds annealing at 60° C., and 30 seconds extension at 72° C.
2 minutes at 72° C. (final extension step)

The isolated genomic DNA of a spinach plant of variety Viroflay comprising the beta-WOLF 0 allele was used in polymerase chain reactions (PCR), using forward primer TCACGTGGGTTGTGTTGT (SEQ ID NO: 7) and reverse primer TTCGCCCTCATCTTCCTGG (SEQ ID NO: 6). The primer pair amplifies the LRR domain-encoding region of a beta-WOLF gene, and has been designed for selectively amplifying part of a WOLF gene, and not of other CC-NBS-LRR protein-encoding genes.

PCR conditions for amplifying the LRR domain-encoding region of a beta-WOLF gene using primers having SEQ ID NO: 6 and SEQ ID NO: 7 were as follows, using Platinum Taq enzyme (Thermo Fisher Scientific):
3 minutes at 95° C. (initial denaturing step)
40 amplification cycles, each cycle consisting of: 30 seconds denaturation at 95° C., 50 seconds annealing at 58° C. and 50 seconds extension at 72° C.
2 minutes at 72° C. (final extension step)

The PCR products were visualized on agarose gel (not shown), and DNA was purified from the PCR reaction. Subsequently the sequence of the PCR products was determined using methods well known in the art.

The DNA sequence of the LRR domain of the alpha-WOLF 23 allele amplified by primers having SEQ ID NO: 5 and SEQ ID NO: 6 is provided in Table 2 under SEQ ID NO: 12.

The DNA sequence of the LRR domain of the beta-WOLF 0 allele amplified by primers having SEQ ID NO: 6 and SEQ ID NO: 7 is provided in Table 2 under SEQ ID NO: 8.

Finally, the obtained sequences were translated into the corresponding amino acid sequence of the LRR domain having SEQ ID NO: 13 and SEQ ID NO: 9 for the alpha-WOLF 23 allele and the beta-WOLF 0, respectively (See also Table 2).

### Example 4

### Introducing an Alpha-WOLF 23 Allele in a Plant not Carrying the Allele

A spinach plant comprising the alpha-WOLF 23 allele was crossed with a plant of variety Viroflay carrying the beta-WOLF 0 allele to obtain an F1 generation. Subsequently, an F1 plant was selfed to obtain an F2 population. Viroflay is susceptible to both CMV and Peronospora effusa.

Plants of the F2 population were assayed as described in Example 1 for resistance to CMV. Approximately 75% of the plants scored completely resistant in the assay. This segregation pattern is consistent with that of a dominant inheritance, which is similar for the downy mildew resistance conferred by this allele.

Genomic DNA of each plant of the same F2 population was isolated and used in two different polymerase chain reactions (PCR). The first PCR reaction was done using primers for amplifying the LRR domain of an alpha-WOLF allele and the second PCR reaction was done using primers for amplifying the LRR domain of a beta-WOLF allele, both as described in Example 3.

The PCR products were visualized on agarose gel (not shown), this demonstrated that approximately 75% of the plants contained an alpha-WOLF fragment, and that the remaining approximately 25% of the plants only contained a beta-WOLF fragment. The plants containing the alpha-WOLF fragment completely correlated with the plants that scored resistant to CMV. The plants only comprising the beta-WOLF fragment completely correlated with the plants that scored susceptible to CMV.

DNA from the PCR reaction was purified, and subsequently the sequence of the PCR products was determined. The alpha-WOLF PCR products gave a sequence that corresponded to the sequence of SEQ ID NO: 12, the sequence of the LRR domain of the alpha-WOLF 23 allele. The beta-WOLF PCR products gave a sequence that corresponded to the sequence of SEQ ID NO: 8 the sequence of the LRR domain of the beta-WOLF 0 allele.

The invention is further described by the following numbered paragraphs:
1. A spinach plant comprising an allele of an alpha-WOLF gene encoding a protein which confers resistance to Cucumber Mosaic Virus (CMV), wherein the protein comprises in its amino acid sequence the motif MAEIGYSVC (SEQ ID NO: 1) at its N-terminus, and the motif KWMCLR (SEQ ID NO: 2); and wherein said allele comprises:
a) a nucleotide sequence comprising a coding sequence having at least 97% sequence identity to SEQ ID NO: 10, or
b) a nucleotide sequence encoding a protein having an amino acid sequence having at least 93.5% sequence identity to SEQ ID NO: 11, or
c) a nucleotide sequence encoding an LRR domain, wherein the nucleotide sequence has at least 94.5% sequence identity to SEQ ID NO: 12, or
d) a nucleotide sequence encoding an LRR domain, wherein the LRR domain comprises an amino acid sequence having at least 93% sequence similarity to SEQ ID NO: 13,
wherein said plant does not comprise a second alpha-WOLF allele having a nucleotide comprising a coding sequence according to SEQ ID NO: 14.
2. The spinach plant of paragraph 1, wherein the said allele further provides resistance to at least Peronospora effusa races Pe:3, Pfs:5, Pe:8, Pe:9, Pe:11, Pe:14, Pe:16 and Pe:19 when homozygously present in a spinach plant.
3. The spinach plant of paragraph 1, wherein the allele of the alpha-WOLF gene is as comprised in the genome of a spinach plant, representative sample of seed of which was deposited with the NCIMB under accession number NCIMB 44747.
4. The spinach plant of paragraph 1, wherein the spinach plant is an agronomically elite spinach plant.
5. The spinach plant of paragraph 4, wherein the agronomically elite spinach plant is a hybrid variety or an inbred line.
6. A seed of, or from, or that produces the spinach plant of paragraph 1 and comprises the allele of the alpha-WOLF gene encoding the protein which confers resistance to CMV.
7. Propagation material from or suitable for producing spinach plant of paragraph 1, wherein the propagation material is suitable for sexual reproduction, and a microspore, pollen, ovary, ovule, embryo sac or an egg cell, or the propagation material is suitable for vegetative reproduction and comprises a cutting, root, stem cell, or a protoplast, or the propagation material is suitable for tissue culture of regenerable cells or protoplasts and comprises a leaf, pollen, embryo, cotyledon, hypocotyl, meristematic cell, root, root tip, anther, flower or a stem, and wherein the propagation material comprises allele of the alpha-WOLF gene encoding the protein which confers resistance to CMV.
8. A method for producing a spinach plant resistant to CMV comprising the steps of:
   a) crossing a first parent plant, wherein the first parent plant is the spinach plant of paragraph 1, with a second parent plant to obtain an F1 population;
   b) optionally performing one or more rounds of selfing and/or crossing with a plant from the F1 population to obtain a further generation population;
   c) selecting from the first or the further generation population a plant resistant to CMV.
9. A method for producing a hybrid spinach seed resistant to CMV, comprising the steps of crossing a first parent plant with a second parent plant, wherein one or both parent plants are homozygous for the allele of the alpha-WOLF gene as described in paragraph 1 and harvesting the hybrid seed.
10. The hybrid seed produced by the method of paragraph 9.
11. A plant grown from the hybrid seed of paragraph 10.
12. A method for growing a spinach plant at least resistant to CMV, comprising the step of sowing or planting the seed of paragraph 6, a representative sample of which was deposited with the NCIMB under accession number NCIMB 44747.
13. The spinach plant of paragraph 1, wherein the allele of the alpha-WOLF gene comprises the nucleotide sequence comprising the coding sequence having at least 97% sequence identity to SEQ ID NO: 10.
14. The spinach plant of paragraph 1, wherein the allele of the alpha-WOLF gene comprises the nucleotide sequence encoding the protein which has the amino acid sequence having at least 93.5% sequence identity to SEQ ID NO: 11.
15. The spinach plant of paragraph 1, wherein the allele of the alpha-WOLF gene comprises the nucleotide sequence encoding the LRR domain, wherein the nucleotide sequence has at least 94.5% sequence identity to SEQ ID NO: 12.
16. The spinach plant of paragraph 1, wherein the allele of the alpha-WOLF gene comprises the nucleotide sequence encoding the LRR domain, wherein the LRR domain comprises the amino acid sequence having at least 93% sequence similarity to SEQ ID NO: 13.
17. A method for selecting a spinach plant resistant to Cucumber Mosaic Virus (CMV), comprising
a) identifying by genetic analysis the presence of an allele of the alpha-WOLF gene encoding a protein which confers resistance to CMV; and
b) selecting a plant that comprises said allele of the alpha-WOLF gene;
wherein:
   the allele of the alpha-WOLF gene comprises:
i) a nucleotide sequence comprising a coding sequence having at least 97% sequence identity to SEQ ID NO: 10; or
ii) a nucleotide sequence encoding a protein which has an amino acid sequence having at least 93.5% sequence identity to SEQ ID NO: 11; or
iii) a nucleotide sequence encoding an LRR domain wherein the nucleotide sequence has at least 94.5% sequence identity to SEQ ID NO: 12; or
iv) a nucleotide sequence encoding an LRR domain, wherein the LRR domain comprises an amino acid sequence having at least 93% sequence similarity to SEQ ID NO: 13;
wherein the protein comprises in its amino acid sequence the motif MAEIGYSVC (SEQ ID NO: 1) at its N-terminus, and the motif KWMCLR (SEQ ID NO: 2); and
wherein the plant selected in b) does not comprise a second alpha-WOLF allele having a nucleotide comprising a coding sequence according to SEQ ID NO: 14.
18. The method of paragraph 17, further comprising the steps of:
c) further testing the selected plant for CMV resistance; and
d) selecting the further tested plant that exhibits CMV resistance.
19. A method for identifying a spinach plant comprising an allele of an alpha-WOLF gene encoding a protein which confers resistance to Cucumber Mosaic Virus (CMV), wherein:
the allele of the alpha-WOLF gene comprises:
i) a nucleotide sequence comprising a coding sequence having at least 97% sequence identity to SEQ ID NO: 10 (nucleotide sequence (i)); or
ii) a nucleotide sequence encoding a protein which has an amino acid sequence having at least 93.5% sequence identity to SEQ ID NO: 11 (nucleotide sequence (ii)); or
iii) a nucleotide sequence encoding an LRR domain wherein the nucleotide sequence has at least 94.5% sequence identity to SEQ ID NO: 12 (nucleotide sequence (iii)); or
iv) a nucleotide sequence encoding an LRR domain, wherein the LRR domain comprises an amino acid sequence having at least 93% sequence identity to SEQ ID NO: 13 (nucleotide sequence (iv)); and
the protein encoded by the allele of the alpha-WOLF gene comprises in its amino acid sequence the motif MAEIGYSVC (SEQ ID NO: 1) at its N-terminus, and the motif KWMCLR (SEQ ID NO: 2);
the method comprises:
detecting by genetic analysis in the genome of the spinach plant nucleotide (i), or nucleotide (ii), nucleotide (iii), or nucleotide (iv), or a unique polymorphism in the allele of the alpha-WOLF gene, thereby identifying the spinach plant comprising the allele of the alpha-WOLF gene;
and optionally testing the plant identified as comprising the allele of the alpha-WOLF gene for exhibiting resistance to CMV;
wherein the plant identified by the method does not comprise a second alpha-WOLF allele having a nucleotide comprising a coding sequence according to SEQ ID NO: 14.
20. The method of paragraph 19, wherein the method comprises determining the presence of the nucleotide sequence encoding the LRR domain by using a primer pair comprising a forward primer and a reverse primer to amplify the nucleotide sequence encoding the LRR domain, wherein the forward primer is a nucleic acid molecule having the sequence of SEQ ID NO: 5 and the reverse primer is a nucleic acid molecule having the sequence of SEQ ID NO: 6.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A spinach plant comprising an allele of an alpha-WOLF gene encoding a protein which confers resistance to Cucumber Mosaic Virus (CMV), wherein the protein comprises in its amino acid sequence the motif MAEIGYSVC (SEQ ID NO: 1) at its N-terminus, and the motif KWMCLR (SEQ ID NO: 2); and wherein said allele comprises:
a) a nucleotide sequence comprising a coding sequence having at least 97% sequence identity to SEQ ID NO: 10, or
b) a nucleotide sequence encoding a protein having an amino acid sequence having at least 93.5% sequence identity to SEQ ID NO: 11, or
c) a nucleotide sequence encoding an LRR domain, wherein the nucleotide sequence has at least 94.5% sequence identity to SEQ ID NO: 12, or
d) a nucleotide sequence encoding an LRR domain, wherein the LRR domain comprises an amino acid sequence having at least 93% sequence similarity to SEQ ID NO: 13,
wherein said plant does not comprise a second alpha-WOLF allele having a nucleotide comprising a coding sequence according to SEQ ID NO: 14.

2. The spinach plant of claim 1, wherein the said allele further provides resistance to at least Peronospora effusa races Pe:3, Pfs:5, Pe:8, Pe:9, Pe:11, Pe:14, Pe:16 and Pe:19 when homozygously present in a spinach plant.

3. The spinach plant of claim 1, wherein the allele of the alpha-WOLF gene is as comprised in the genome of a spinach plant, representative sample of seed of which was deposited with the NCIMB under accession number NCIMB 44747.

4. The spinach plant of claim 1, wherein the spinach plant is an agronomically elite spinach plant.

5. The spinach plant of claim 4, wherein the agronomically elite spinach plant is a hybrid variety or an inbred line.

6. A seed of, or from, or that produces the spinach plant of claim 1 and comprises the allele of the alpha-WOLF gene encoding the protein which confers resistance to CMV.

7. Propagation material from or suitable for producing spinach plant of claim 1, wherein the propagation material is suitable for sexual reproduction, and a microspore, pollen, ovary, ovule, embryo sac or an egg cell, or the propagation material is suitable for vegetative reproduction and comprises a cutting, root, stem cell, or a protoplast, or the propagation material is suitable for tissue culture of regenerable cells or protoplasts and comprises a leaf, pollen, embryo, cotyledon, hypocotyl, meristematic cell, root, root tip, anther, flower or a stem, and wherein the propagation material comprises allele of the alpha-WOLF gene encoding the protein which confers resistance to CMV.

8. A method for producing a spinach plant resistant to CMV comprising the steps of:
a) crossing a first parent plant, wherein the first parent plant is the spinach plant as claimed in claim 1, with a second parent plant to obtain an F1 population;
b) optionally performing one or more rounds of selfing and/or crossing with a plant from the F1 population to obtain a further generation population;
c) selecting from the first or the further generation population a plant resistant to CMV.

9. A method for producing a hybrid spinach seed resistant to CMV, comprising the steps of crossing a first parent plant with a second parent plant, wherein one or both parent plants are homozygous for the allele of the alpha-WOLF gene as described in claim 1 and harvesting the hybrid seed.

10. The hybrid seed produced by the method of claim 9.

11. A plant grown from the hybrid seed of claim 10.

12. A method for growing a spinach plant at least resistant to CMV, comprising the step of sowing or planting the seed of claim 6, a representative sample of which was deposited with the NCIMB under accession number NCIMB 44747.

13. The spinach plant of claim 1, wherein the allele of the alpha-WOLF gene comprises the nucleotide sequence comprising the coding sequence having at least 97% sequence identity to SEQ ID NO: 10.

14. The spinach plant of claim 1, wherein the allele of the alpha-WOLF gene comprises the nucleotide sequence encoding the protein which has the amino acid sequence having at least 93.5% sequence identity to SEQ ID NO: 11.

15. The spinach plant of claim 1, wherein the allele of the alpha-WOLF gene comprises the nucleotide sequence encoding the LRR domain, wherein the nucleotide sequence has at least 94.5% sequence identity to SEQ ID NO: 12.

16. The spinach plant of claim 1, wherein the allele of the alpha-WOLF gene comprises the nucleotide sequence encoding the LRR domain, wherein the LRR domain comprises the amino acid sequence having at least 93% sequence similarity to SEQ ID NO: 13.

17. A method for selecting a spinach plant resistant to Cucumber Mosaic Virus (CMV), comprising
a) identifying by genetic analysis the presence of an allele of the alpha-WOLF gene encoding a protein which confers resistance to CMV; and
b) selecting a plant that comprises said allele of the alpha-WOLF gene;
wherein:
the allele of the alpha-WOLF gene comprises:
i) a nucleotide sequence comprising a coding sequence having at least 97% sequence identity to SEQ ID NO: 10; or
ii) a nucleotide sequence encoding a protein which has an amino acid sequence having at least 93.5% sequence identity to SEQ ID NO: 11; or
iii) a nucleotide sequence encoding an LRR domain wherein the nucleotide sequence has at least 94.5% sequence identity to SEQ ID NO: 12; or
iv) a nucleotide sequence encoding an LRR domain, wherein the LRR domain comprises an amino acid sequence having at least 93% sequence similarity to SEQ ID NO: 13;
wherein the protein comprises in its amino acid sequence the motif MAEIGYSVC (SEQ ID NO: 1) at its N-terminus, and the motif KWMCLR (SEQ ID NO: 2); and
wherein the plant selected in b) does not comprise a second alpha-WOLF allele having a nucleotide comprising a coding sequence according to SEQ ID NO: 14.

18. The method of claim 17, further comprising the steps of:
c) further testing the selected plant for CMV resistance; and
d) selecting the further tested plant that exhibits CMV resistance.

19. A method for identifying a spinach plant comprising an allele of an alpha-WOLF gene encoding a protein which confers resistance to Cucumber Mosaic Virus (CMV), wherein:
the allele of the alpha-WOLF gene comprises:
i) a nucleotide sequence comprising a coding sequence having at least 97% sequence identity to SEQ ID NO: 10 (nucleotide sequence (i)); or
ii) a nucleotide sequence encoding a protein which has an amino acid sequence having at least 93.5% sequence identity to SEQ ID NO: 11 (nucleotide sequence (ii)); or
iii) a nucleotide sequence encoding an LRR domain wherein the nucleotide sequence has at least 94.5% sequence identity to SEQ ID NO: 12 (nucleotide sequence (iii)); or
iv) a nucleotide sequence encoding an LRR domain, wherein the LRR domain comprises an amino acid sequence having at least 93% sequence identity to SEQ ID NO: 13 (nucleotide sequence (iv)); and
the protein encoded by the allele of the alpha-WOLF gene comprises in its amino acid sequence the motif MAEIGYSVC (SEQ ID NO: 1) at its N-terminus, and the motif KWMCLR (SEQ ID NO: 2);
the method comprises:
detecting by genetic analysis in the genome of the spinach plant nucleotide (i), or nucleotide (ii), nucleotide (iii), or nucleotide (iv), or a unique polymorphism in the allele of the alpha-WOLF gene, thereby identifying the spinach plant comprising the allele of the alpha-WOLF gene;
and optionally testing the plant identified as comprising the allele of the alpha-WOLF gene for exhibiting resistance to CMV;
wherein the plant identified by the method does not comprise a second alpha-WOLF allele having a nucleotide comprising a coding sequence according to SEQ ID NO: 14.

20. The method of claim 19, wherein the method comprises determining the presence of the nucleotide sequence encoding the LRR domain by using a primer pair comprising a forward primer and a reverse primer to amplify the nucleotide sequence encoding the LRR domain, wherein the forward primer is a nucleic acid molecule having the sequence of SEQ ID NO: 5 and the reverse primer is a nucleic acid molecule having the sequence of SEQ ID NO: 6.
